(19) United States
(12) Patent Application Publication
(10) Pub. No.: US 2026/0124184 A1
(43) Pub. Date: May 7, 2026

(54) ONCE DAILY ALDOSTERONE SYNTHASE INHIBITOR (R)-(+)-5-(P-CYANOPHENYL)-5,6,7,8-TETRAHYDROIMIDAZO[L,5-A]PYRIDINE

(71) Applicant: DAMIAN PHARMA AG, Walchwil (CH)

(72) Inventors: Christoph SCHUMACHER, Walchwil (CH); Ronald Edward STEELE, Long Valley, NJ (US); Teresa GERLOCK, Walchwil (CH)

(21) Appl. No.: 19/119,988

(22) PCT Filed: Oct. 13, 2023
(86) PCT No.: PCT/EP2023/078505

§ 371 Date: Apr. 10, 2025

(30) Foreign Application Priority Data
Oct. 14, 2022 (EP) 22201751.9

(51) Int. Cl. A61K 31/437; A61P 5/42; A61P 7/00; A61P 9/12

(52) U.S. Cl. CPC A61K 31/437; A61P 5/42; A61P 7/00; A61P 9/12

(57) ABSTRACT

The present invention relates to a composition for use in the treatment of a disease or disorder, wherein said disease or disorder is preferably a disease or disorder in which aldosterone overexposure contributes to the symptoms of said disease or disorder, said composition comprises a compound which is (R)-(+)-5-(p-cyanophenyl)-5,6,7,8-tetrahydroimidazo[1,5-a]pyridine or a pharmaceutically acceptable salt thereof, wherein preferably said compound is (R)-(+)-5-(p-cyanophenyl)-5,6,7,8-tetrahydroimidazolium[1,5-a]pyridine dihydrogen phosphate, and wherein said compound has an enantiomeric excess (ee) of the (R) form higher than or equal to 99%, and wherein said composition is administered once daily for at least eight weeks to a patient in need thereof.

The present invention relates to a composition for use in the treatment of a disease or disorder, wherein said disease or disorder is preferably a disease or disorder in which aldosterone overexposure contributes to the symptoms of said disease or disorder, said composition comprises a compound which is (R)-(+)-5-(p-cyanophenyl)-5,6,7,8-tetrahydroimidazo[1,5-a]pyridine or a pharmaceutically acceptable salt thereof, wherein preferably said compound is (R)-(+)-5-(p-cyanophenyl)-5,6,7,8-tetrahydroimidazolium[1,5-a]pyridine dihydrogen phosphate, and wherein said compound has an enantiomeric excess (ee) of the (R) form higher than or equal to 99%, and wherein said composition is administered once daily for at least eight weeks to a patient in need thereof. Diseases of aldosterone overexposure can be diagnosed biochemically and include in decreasing severity primary aldosteronism due to adrenal aldosterone-producing adenoma or nodules or due to adrenal hyperplasia or due to apparently normal glands.

### RELATED ART

Aldosterone excess is associated with or can accompany various harmful metabolic and cardiovascular structural effects, including sodium and water retention, hypokalemia, hypomagnesaemia, hypertension, left ventricular hypertrophy and cardiac fibrosis. Primary aldosteronism has a substantially adverse outcome when compared with essential hypertension. Accordingly, effective medical therapy both to lower blood pressure and antagonize the excess activation of the mineralocorticoid receptor are important therapeutic aims (Parthasarathy et al., 2011, J Hypertens 29: 980-990).

Primary aldosteronism (PA) is defined as a group of disorders in which aldosterone production is inappropriately high for sodium status, relatively autonomous of the major regulators of secretion (angiotensin II, plasma potassium concentration), and non-suppressible by sodium loading. Such inappropriate production of aldosterone causes hypertension, cardiovascular damage, sodium retention, suppression of plasma renin, and increased potassium excretion that (if prolonged and severe) may lead to hypokalemia. PA is commonly caused by a benign adrenal adenoma, adrenal nodules, unilateral or bilateral adrenal hyperplasia (BAH), or in rare cases adrenal carcinoma or inherited conditions of familial hyperaldosteronism. PA is also known as Conn's syndrome (Funder et al., 2016, J Clin Endocrinol Metab 101: 1889-1916).

Aldosterone levels can be measured in the blood and in the urine. The Endocrine Society clinical practice guidelines for the management of primary aldosteronism describe in detail the process and methods to measure the aldosterone-to-renin ratio (ARR) in blood samples (Funder et al., 2016, J Clin Endocrinol Metab 101: 1889-1916). The guidelines also recommend diagnostic cut-off levels for abnormal values. A more accurate method to measure aldosterone levels consist of the collection of a 24-hour urine sample and determination of the content of tetrahydroaldosterone, the main metabolite of aldosterone, in the urine (Funder and Carey, 2022, Hypertens 79: 726-735). The authors provide diagnostic cut-off values for abnormal values.

Furthermore, the Endocrine Society clinical practice guidelines for the management of primary aldosteronism recommend and describe an invasive process supported by CT imaging to subtype patients to lateralize the adrenal source of excessive aldosterone secretion. Unilateral adrenal adenoma can be surgically removed by adrenalectomy whereas bilateral disease requires medical treatment (Funder et al., 2016, J Clin Endocrinol Metab 101: 1889-1916).

The capacity of adrenal glomerulosa cells to produce aldosterone is controlled largely by the regulated transcription of CYP11B2, the gene encoding aldosterone synthase. Aldosterone synthase inhibition has emerged as an option for the treatment of hypertension, heart failure and renal disorders. The aim is to decrease aldosterone concentrations in both plasma and tissues, thereby decreasing MR-dependent and MR-independent effects in the cardiac, vascular and renal target organs.

(R)-(+)-5-(p-cyanophenyl)-5,6,7,8-tetrahydroimidazolium[1,5-a]pyridine is a small molecule capable of inhibiting aldosterone synthase. WO 2018/078049 teaches the preparation of (R)-(+)-5-(p-cyanophenyl)-5,6,7,8-tetrahydroimidazolium[1,5-a]pyridine and pharmaceutically acceptable salts thereof (e.g., (R)-(+)-5-(p-cyanophenyl)-5,6,7,8-tetrahydroimidazolium[1,5-a]pyridine dihydrogen phosphate; also known as dexfadrostat phosphate) at an enantiomeric excess (ee) of the (R) form higher than or equal to 99%. The high enantiomeric excess values achieved by the process described in WO 2018/078049 leads to potent (i.e., low nanomolar) and selective aldosterone synthase inhibition by (R)-(+)-5-(p-cyanophenyl)-5,6,7,8-tetrahydroimidazolium[1,5-a]pyridine.

WO 2019/211394 teaches the results of a phase 1 clinical trial investigating the use of dexfadrostat phosphate in healthy subjects.

### SUMMARY OF THE INVENTION

In a prospective, double-blind, randomized, Phase 2, efficacy study in patients diagnosed with primary aldosteronism, the inventors have surprisingly found that a preferred compound of the present invention was able to effectively normalize the aldosterone-to-renin ratio in said patients compared to baseline values by suppressing aldosterone synthesis at a once daily dose of 4 mg. This finding is particularly remarkable since the preceding clinical phase 1 study in healthy subjects with normal aldosterone levels indicated limited aldosterone-suppressing potency of the 4 mg dose once daily.

Furthermore, the inventors have surprisingly shown that a preferred compound of the present invention was able to normalize plasma aldosterone levels in said patients at once daily doses of 4 mg, 8 mg, and 12 mg independently of the causal adrenal pathology such as unilateral or bilateral adenoma, unilateral or bilateral nodules, unilateral or bilateral hyperplasia as well as normal appearing adrenal glands. This finding is particularly remarkable since the patients with primary aldosteronism due to adenoma are characterized by extreme aldosterone and potassium levels requiring adrenal surgery for management.

Furthermore, the inventors have surprisingly observed that a preferred compound of the present invention was able to normalize plasma aldosterone levels in said patients at once daily dose of 4 mg, 8 mg, and 12 mg with a rapid response i.e., within a short treatment duration of 14 days or less that translated to rapid normalization of blood pressure and hypokalemia. This finding is particularly remarkable since clinical responses to surgery and current medical treatment can take 3 to 12 months to occur.

Furthermore, the inventors have surprisingly observed that a preferred compound of the present invention was able to normalize plasma aldosterone levels in said patients at a once daily dose of 4 mg, 8 mg, and 12 mg with a rapid response that was sustained over the entire treatment duration without dose adjustment and which translated to sustained normalization of blood pressure and hypokalemia indicative of long-term cardiovascular protection. This finding is particularly remarkable since current medical treatment requires biweekly dose-titration to balance efficacy and tolerability.

Furthermore, the inventors have surprisingly observed that a preferred compound of the present invention was able to normalize urinary aldosterone secretion in said patients at a once daily dose of 4 mg, 8 mg, and 12 mg expanding the utility of the compound to diseases characterized by abnormal aldosterone content into the urine such as essential hypertension, resistant hypertension and heart and renal failure. This finding is particularly remarkable since diseases since current medical treatments (e.g. mineralcorticoid receptor antagonists) further induce aldosterone production as a consequence of treatment.

In one aspect, the present invention provides a composition for use in the treatment of a disease or disorder, wherein said composition is administered once daily to a subject in need thereof for at least eight weeks and wherein said composition comprises a compound which is (R)-(+)-5-(p-cyanophenyl)-5,6,7,8-tetrahydroimidazo[1,5-a]pyridine or a pharmaceutically acceptable salt thereof, wherein said compound has an enantiomeric excess (ee) of the (R) form higher than or equal to about 99%.

Accordingly, in one aspect, the present invention provides for a composition for use in the treatment of a disease or disorder, said composition comprises a compound which is (R)-(+)-5-(p-cyanophenyl)-5,6,7,8-tetrahydroimidazo[1,5-a]pyridine or dexfadrostat (formula (I)) or a pharmaceutically acceptable salt thereof, wherein said compound has an enantiomeric excess (ee) of the (R) form higher than or equal to 99%, and wherein said composition is administered once daily to a patient in need thereof at a dose of 1-8 mg.

In a further aspect, the present invention provides for a composition for use in the treatment of a disease or disorder, wherein said disease or disorder is preferably primary aldosteronism, further preferably primary aldosteronism of various pathological subtypes such as unilateral or bilateral adenoma, unilateral or bilateral nodules, unilateral of bilateral adrenal hyperplasia as well as normal appearing adrenal glands.

In a further aspect, the present invention provides for a composition for use in the treatment of a disease or disorder, wherein said disease or disorder is primary aldosteronism in which aldosterone overexposure contributes to debilitating symptoms and events, and patients require a rapid correction of the underlying biochemical (hyperaldosteronism, hypokalemia) and clinical (systolic and diastolic blood pressure) health risk hazards.

In a further aspect, the present invention provides for a composition for use in the treatment of a disease or disorder, wherein said disease or disorder is primary aldosteronism in which aldosterone overexposure contributes to debilitating symptoms and events, and patients require a sustained and reliable correction of the underlying biochemical (hyperaldosteronism, hypokalemia) and clinical (systolic and diastolic blood pressure) health risk hazards.

In a further aspect, the present invention provides for composition for use in the treatment of a disease or disorder, wherein said disease or disorder is characterized by abnormal or inappropriate aldosterone content in the urine such as primary aldosteronism, secondary aldosteronism, heart failure, renal failure, resistant hypertension, essential hypertension, nephropathy, edema, coronary heart disease, hypokalemia, and cardiac arrhythmias.

In one aspect, the present invention provides a composition for use in the treatment of a disease or disorder, wherein said composition is administered once daily to a subject, preferably a human, in need thereof wherein said composition comprises a compound which is (R)-(+)-5-(p-cyanophenyl)-5,6,7,8-tetrahydroimidazo[1,5-a]pyridine or a pharmaceutically acceptable salt thereof, wherein said compound has an enantiomeric excess (ee) of the (R) form higher than or equal to about 99%.

In some embodiments, said composition is administered once daily to a subject in need thereof for at least eight weeks.

In some embodiments, said disease or disorder is a chronic disease or disorder, preferably a chronic cardiovascular disease or disorder.

In some embodiments, said composition is administered once daily to said subject at a dosage from about 1 mg to about 12 mg of said compound. In some embodiments, said compound is administered at a dosage of about 4 mg. In some embodiments, said compound is administered at a dosage of about 8 mg.

In some embodiments, said compound is (R)-(+)-5-(p-cyanophenyl)-5,6,7,8-tetrahydroimidazolium[1,5-a]pyridine dihydrogen phosphate, preferably wherein said (R)-(+)-5-(p-cyanophenyl)-5,6,7,8-tetrahydroimidazolium[1,5-a]pyridine dihydrogen phosphate has an enantiomeric excess (ee) of the (R) form higher than or equal to about 99.5%, more preferably higher than or equal to about 99.9%.

In some embodiments, said disease or disorder is selected from the group consisting of primary aldosteronism, secondary aldosteronism, heart failure, chronic renal failure, hypertension, restenosis, obesity, nephropathy, post-myocardial infarction, renal fibrosis, coronary heart disease, sodium retention, water retention, hypokalemia, hypomagnesaemia, hypertension, left ventricular hypertrophy, cardiac fibrosis, cardiovascular damage, suppression of plasma renin, kidney fibrosis, arrhythmias, nephropathy, edema, hypokalemia-caused muscle weakness, cardiac fibrillation and weakened cardiac muscle contraction; preferably wherein said disease or disorder is selected from the group consisting of primary aldosteronism, secondary aldosteronism, heart failure, chronic renal failure, hypertension, restenosis, obesity, nephropathy, post-myocardial infarction, renal fibrosis, coronary heart disease, sodium retention, water retention, hypokalemia, hypomagnesaemia, hypertension, left ventricular hypertrophy, cardiac fibrosis, cardiovascular damage, suppression of plasma renin, kidney fibrosis, arrhythmias, nephropathy, and edema.

In some embodiments, said disease or disorder is primary aldosteronism.

In some embodiments, said disease or disorder is bilateral primary aldosteronism.

In some embodiments, said disease or disorder is unilateral primary aldosteronism, preferably wherein said unilateral primary aldosteronism is caused by unilateral adenoma, unilateral nodules, or unilateral hyperplasia.

In some embodiments, said disease or disorder is essential hypertension and/or resistant hypertension, preferably wherein said disease or disorder is essential hypertension and/or resistant hypertension with accompanying hypokalemia.

In some embodiments, said disease or disorder is hypokalemia.

In some embodiments, said once daily dose does not require dose adjustment.

In some embodiments, said subject exhibits a clinical response within about 14 days of treatment, preferably wherein said clinical response is selected from reduction in plasma aldosterone levels, normalization of plasma aldosterone levels, reduction of blood pressure, normalization of blood pressure, reduction of hypokalemia, normalization of potassium concentration, and reduction of urinary THA.

It is believed that the excessive increase of aldosterone production in primary aldosteronism is a consequence of an escape of clusters of cells in the adrenal glomerulosa from the normal physiological control. It is further believed thus that an aldosterone synthase inhibitor has the potential to prevent the sequalae of excessive aldosterone synthesis if provided at appropriate doses with appropriate duration of activity. In a preferred embodiment, said disease or disorder treated according to the invention is a condition following or occurring as a consequence of aldosterone overexposure.

### DESCRIPTION OF THE FIGURES

FIG. 1 is a schematic of study design the phase II clinical trial described in Example 1.

FIG. 2A is a plot of aldosterone to renin ratio (ARR) (ng/dL)/(mU/L) of all patients taken at Day 1 and Day 56. LSM change in lognormal values from baseline was −2.54 (95% CI −2.9 to −2.2). *p<0.0001.

FIG. 2B is a plot of mean (SEM) 24-hour ambulatory SBP (aSBP) (mmHg) of all patients taken at Day 1 and Day 56. LSM change from baseline was −10.7 mmHg (95% CI −13.6 to −7.9). *p<0.0001.

FIG. 3A is a plot of aldosterone to renin ratio (ARR) (ng/dL)/(mU/L) of patients with unilateral PA taken at Day 1, Day 14, Day 28, Day 42 and Day 56.

FIG. 3B is a plot of aldosterone to renin ratio (ARR) (ng/dL)/(mU/L) of patients with bilateral PA or PA with unknown cause (idiopathic PA) taken at Day 1, Day 14, Day 28, Day 42 and Day 56.

FIG. 4A is a plot of LSM change in lognormal values for aldosterone to renin ratio (ARR) (ng/dL)/(mU/L) in patients treated with 4 mg dexafadrostat (n=10) taken at Day 1 and Day 56. Box plots show medians, first and third quartiles, and ranges. *p<0.0005.

FIG. 4B is a plot of LSM change in lognormal values for aldosterone to renin ratio (ARR) (ng/dL)/(mU/L) in patients treated with 8 mg dexafadrostat (n=12) taken at Day 1 and Day 56. Box plots show medians, first and third quartiles, and ranges. *p<0.0005.

FIG. 4C is a plot of LSM change in lognormal values for aldosterone to renin ratio (ARR) (ng/dL)/(mU/L) in patients treated with 12 mg dexafadrostat (n=13) taken at Day 1 and Day 56. Box plots show medians, first and third quartiles, and ranges. *p<0.0005.

FIG. 4D is a plot of LSM change in 24-hour ambulatory SBP (aSBP) (mmHg) in patients treated with 4 mg dexafadrostat (n=10) taken at Day 1 and Day 56. *p<0.0005.

FIG. 4E is a plot of LSM change in 24-hour ambulatory SBP (aSBP) (mmHg) in patients treated with 8 mg dexafadrostat (n=12) taken at Day 1 and Day 56. *p<0.0005.

FIG. 4F is a plot of LSM change in 24-hour ambulatory SBP (aSBP) (mmHg) in patients treated with 8 mg dexafadrostat (n=13) taken at Day 1 and Day 56. *p<0.0005.

FIG. 4G is a plot of LSM change in urinary tetrahydroaldosterone (THA) (μg/24 h) in patients treated with 4 mg dexafadrostat (n=10) taken at Day 1 and Day 56. Box plots show medians, first and third quartiles, and ranges. *p<0.0001.

FIG. 4H is a plot of LSM change in urinary tetrahydroaldosterone (THA) (μg/24 h) in patients treated with 8 mg dexafadrostat (n=12) taken at Day 1 and Day 56. Box plots show medians, first and third quartiles, and ranges. *p<0.0001.

FIG. 41 is a plot of LSM change in urinary tetrahydroaldosterone (THA) (μg/24 h) in patients treated with 12 mg dexafadrostat (n=13) taken at Day 1 and Day 56. Box plots show medians, first and third quartiles, and ranges. *p<0.0001.

FIG. 5A is a plot of aldosterone to renin ratio (ARR) (ng/dL)/(mU/L) measured in LSM change in lognormal values from baseline in patients treated with 4 mg dexafadrostat (n=10) at days 14, 28, 42, 56, and 70. Box plots show medians, first and third quartiles, and ranges. *p≤0.0008.

FIG. 5B is a plot of office systolic blood pressure (oSBP) (mmHg) measured in LSM change from baseline in patients treated with 4 mg dexfadrostat (n=10) at days 14, 28, 42, 56, and 70. *p<0.0008.

FIG. 5C is a plot of mean (SEM) plasma potassium concentration (mmol/L) measured in LSM change from baseline in patients treated with 4 mg dexfadrostat (n=10) at days 14, 28, 42, 56, and 70. *p<0.0008.

FIG. 5D is a plot of aldosterone to renin ratio (ARR) (ng/dL)/(mU/L) measured in LSM change in lognormal values from baseline in patients treated with 8 mg dexafadrostat (n=12) at days 14, 28, 42, 56, and 70. Box plots show medians, first and third quartiles, and ranges. * p<0.0001; ** p<0.05.

FIG. 5E is a plot of office systolic blood pressure (oSBP) (mmHg) measured in LSM change from baseline in patients treated with 8 mg dexfadrostat (n=12) at days 14, 28, 42, 56, and 70. * p<0.0001; ** p<0.05.

FIG. 5F is a plot of mean (SEM) plasma potassium concentration (mmol/L) measured in LSM change from baseline in patients treated with 8 mg dexfadrostat (n=12) at days 14, 28, 42, 56, and 70. * p<0.0001; ** p<0.05.

### DETAILED DESCRIPTION OF THE INVENTION

Unless defined otherwise, all technical and scientific terms used herein have the same meanings as commonly understood by one of ordinary skill in the art to which this invention belongs.

The term “about” where used means especially ±10%, ±5% or ±3% (referring to the given numeric value, respectively), if not indicated otherwise. In each of the invention embodiments, “about” can be deleted. All ranges of values disclosed herein, should refer and include to any and all values falling within said range including the values defining the range. By way of clarification, for example, a value of 12 to 13 should refer to a value of 12 or 13 or any and all values falling within 12 and 13, and for example a daily dosage from 1 mg to 16 mg should refer to a daily dose of 1 mg or 16 mg or any and all values falling within 1 mg and 16 mg.

The term “adenoma” as used herein is a type of non-cancerous or benign tumor that may affect various organs.

The term “adrenal vein sampling” as used herein and abbreviated as “AVS” refers to a procedure used to subtype primary aldosteronism (PA) in combination with computed tomography (CT) scanning. Blood is circulated out of each adrenal gland by an adrenal vein (AV), which ultimately drains into the inferior vena cava (VC; the right AV drains directly into the VC; the left drains into the left renal vein which drains into the inferior VC). AVS is considered the most reliable method to subtype PA.

Intravenous (IV) cannulation is a technique in which a cannula (thin tube) is placed inside a vein to provide venous access. Venous access allows sampling of blood, as well as administration of fluids, medications, parenteral nutrition, chemotherapy, and blood products. The right adrenal vein is the most difficult to cannulate, due to its location and small diameter.

The term “aldosterone-producing cell clusters (APCC)” as used herein refers to cell clusters that strongly express aldosterone synthase (CYP11B2). They are frequently found in adult adrenals and can be found in aldosterone-producing adenomas.

The term “aldosterone synthase” as used herein refers to the steroid hydroxylase cytochrome P450 enzyme CYP11B2. Therefore, aldosterone synthase inhibition is equivalent to CYP11B2 inhibition.

The term “chiral purity” as used herein is defined by the enantiomeric excess (ee) as determined by chiral HPLC (see Examples for details) and calculated by the equation:

$$e e = ( {A}_{R} - {A}_{S} ) / ( {A}_{R} + {A}_{S} ) \times 100 \% ,$$

wherein A_{R} is the area of the (R)-(+)-5-(p-cyanophenyl)-5,6,7,8-tetrahydroimidazo[1,5-a]pyridine peak in the HPLC chromatogram of the sample solution and Aₛ is the area of the (S)-(+)-5-(p-cyanophenyl)-5,6,7,8-tetrahydroimidazo[1,5-a]pyridine peak in the HPLC chromatogram of the sample solution.

The term “disease” and “disorder” are used interchangeably herein, referring to any derangement or abnormal condition or function or morbid physical or mental state, especially an abnormal medical condition such as an illness or injury, wherein a tissue, an organ or an individual is not able to efficiently fulfil its function anymore. See Dorland's Illustrated Medical Dictionary (VSIB, Saunders Co., 27^{th} ed., 1988). Typically, but not necessarily, a disease is associated with or can accompany specific symptoms or signs indicating the presence of such disease. The presence of such symptoms or signs may thus, be indicative for a tissue, an organ or an individual suffering from a disease. Preferably, a tissue, an organ or an individual being at “risk of developing” shows potential of a disease emerging is also covered by the term “disease” or “disorder”. Typically, the risk of developing a disease is associated with early or weak signs or symptoms of such disease.

As used herein, the expression “disease or disorder in which aldosterone overexposure contributes to the symptoms of said disease or disorder” preferably refers to a disease or disorder which is due to the abnormal or inappropriate activity/expression of aldosterone synthase and the biological activity or process which is associated with the abnormal or inappropriate expression of aldosterone synthase. Typical examples of diseases or disorders that are due to abnormal or inappropriate activity/expression of aldosterone synthase are primary aldosteronism, secondary aldosteronism, heart failure, chronic renal failure, hypertension, restenosis, obesity, nephropathy, post-myocardial infarction, renal fibrosis, coronary heart disease, sodium retention, water retention, hypokalemia, hypomagnesaemia, hypertension, left ventricular hypertrophy and cardiac fibrosis, cardiovascular damage, suppression of plasma renin, kidney fibrosis, arrhythmias, nephropathy, edema, and hypokalemia caused muscle weakness, cardiac fibrillation and weakened cardiac muscle contraction. Preferably said diseases or disorders that are due to abnormal or inappropriate activity/expression of aldosterone synthase are primary aldosteronism, secondary aldosteronism, heart failure, chronic renal failure, hypertension, restenosis, obesity, nephropathy, post-myocardial infarction, renal fibrosis, coronary heart disease, sodium retention, water retention, hypokalemia, hypomagnesaemia, hypertension, left ventricular hypertrophy and cardiac fibrosis, cardiovascular damage, suppression of plasma renin, kidney fibrosis, arrhythmias, nephropathy, edema.

The term “nodules” as used herein refers to abnormal tissue growths that can be found anywhere in the body. Although they are often benign, some nodules are symptoms of an underlying health condition.

The term “pharmaceutically acceptable salt” as used herein refers to a salt that is pharmaceutically acceptable and that possesses the desired pharmacological activity of the parent compound. Such salts include acid addition salts formed with inorganic acids or organic acids known to the skilled person in the art (P. Heinrich Stahl (Editor), Camille G. Wermuth (Editor); Handbook of Pharmaceutical Salts: Properties, Selection, and Use, 2nd Revised Edition, March 2011, Wiley-VCH, ISBN: 978-3-90639-051-2). Particularly preferred pharmaceutically acceptable salts in the present invention are acid addition salts, such as hydrochloride or phosphate salts, e.g. formed with phosphoric acid, i.e. a dihydrogen phosphate.

The term “phosphate salt” as used in the present application refers to compounds comprising (R)-(+)-5-(p-cyanophenyl)-5,6,7,8-tetrahydroimidazo[1,5-a]pyridine in its protonated form, i.e. the (R)-(+)-5-(p-cyanophenyl)-5,6,7,8-tetrahydroimidazolium[1,5-a]pyridine cation and further comprising anions derived from phosphoric acid, wherein said anions is typically and preferably dihydrogen phosphate [H₂PO₄] or hydrogen phosphate [HPO₄]²⁻. Preferably, the term “phosphate salt” as used in the present application refers to the dihydrogen phosphate of formula (I), that is, wherein the compound of formula (I) is protonated once and the counterion is [H₂PO₄] and, thus, the stoichiometry of mono-protonated (R)-(+)-5-(p-cyanophenyl)-5,6,7,8-tetrahydroimidazo[1,5-a]pyridine to dihydrogen phosphate is 1:1. The latter compound is referred herein as (R)-(+)-5-(p-cyanophenyl)-5,6,7,8-tetrahydroimidazolium[1,5-a]pyridine dihydrogen phosphate.

The term “primary aldosteronism” and abbreviated as PA is defined herein as a group of disorders characterized by an autonomous aldosterone excess leading to suppression of renin levels. An advocated screening test is the aldosterone-to-renin ratio with pathologically increased values in PA. PA thus covers also disorders in which aldosterone production is inappropriately high for sodium status, relatively autonomous of the major regulators of secretion (angiotensin II, plasma potassium concentration), and non-suppressible by sodium loading. The term “primary aldosteronism” covers also hyperaldosteronism and diseases and disorder which are a consequence of inappropriate production of aldosterone, which are typically and preferably selected from hypertension, cardiovascular damage, sodium retention, water retention, suppression of plasma renin, and increased potassium excretion and hypokalemia, hypomagnesaemia, left ventricular hypertrophy, cardiac fibrosis and kidney fibrosis, heart failure, arrhythmias, nephropathy, edema, and hypokalemia caused muscle weakness, cardiac fibrillation, weakened cardiac muscle contraction and cardiac failure. The term “primary aldosteronism” covers also diseases or disorders that cause PA, which are typically and preferably adrenal adenoma, unilateral and bilateral adrenal hyperplasia (BAH) (e.g. bilateral idiopathic (micronodular) adrenal hyperplasia and primary (unilateral) adrenal hyperplasia), adrenal carcinoma (e.g. aldosterone-producing adrenocortical carcinoma and ectopic aldosterone-producing adenoma or carcinoma), glucocorticoid-remediable aldosteronism, inherited conditions of familial aldosteronism and FH type II. PA is also known as Conn's syndrome. The excessive increase of aldosterone production in PA is a consequence of an escape of clusters of cells in the adrenal glomerulosa from the normal regulatory control of the aldosterone-renin-aldosterone. In some embodiments, hyperaldosteronism is defined as a blood aldosterone concentration of 416 pmol/L (i.e., 15 ng/dL) or higher. In some embodiments, hyperaldosteronism is defined as a urine aldosterone concentration of 10 μg/24 hour or higher. In some embodiments, hyperaldosteronism is defined as a blood aldosterone concentration of 416 pmol/L (i.e., 15 ng/dL) or higher and a urine aldosterone concentration of 10 μg/24 hour or higher. In preferred embodiments, the hyperaldosteronism is blood pressure-independent hyperaldosteronism.

The term “unilateral PA” as used herein refers to a specific subtype of primary aldosteronism wherein only one adrenal gland is the apparent cause of the uncontrolled, overproduction of aldosterone which is the main feature of PA. Unilateral disease is typically caused by an aldosterone producing adenoma (benign tumor) and less commonly by adrenal cancer or hyperplasia (when the whole gland is hyperactive). In preferred embodiments subtyping is done using a combination of computed tomography (CT) scanning and adrenal vein sampling (AVS).

The term “bilateral PA” as used herein refers to a specific subtype of primary aldosteronism wherein both adrenal glands appear to be responsible for the uncontrolled, overproduction of aldosterone which is the main feature of PA. It may be caused by an odd growth in both adrenal glands (known as a bilateral adrenal hyperplasia). In preferred embodiments subtyping is done using a combination of computed tomography (CT) scanning and adrenal vein sampling (AVS).

The terms “hyperplastic PA” or “adrenal hyperplasia” as used herein refer to another form of primary aldosteronism, caused by the abnormal or unusual growth of extra cells within the adrenal gland. Hyperplastic PA can be present in either unilateral or bilateral disease.

The term “idiopathic PA” as used herein refers to primary aldosteronism of unknown cause.

The term “genetic PA” as used herein refers to primary aldosteronism arising from genetic causes. Familial hyperaldosteronism is a heritable cause of PA defined by a family history of two or more affected family members that are not explained by another genetic mechanism.

The term “rapid response” as used herein refers to the body's automatic reaction(s) to a stimulus. A ‘rapid’ response in this case means a response that occurred more quickly than anticipated given the mechanistic pathway of the stimulus and reaction.

The term “sustained response” as used herein refers to a response that stays stable i.e., maintained at length without interruption or weakening, over time. A sustained response is a response that is thus lasting or prolonged.

The term “secondary aldosteronism” (SA) (also hyperreninism, or hyperreninemic hyperaldosteronism) as used herein refers to the hypersecretion of aldosterone secondary to stimulation from sources outside the adrenal gland. Extra-adrenal stimuli are for example renal hypoperfusion. SA is due to overactivity of the renin-angiotensin-aldosterone system. The term “secondary aldosteronism” covers diseases and disorder which are a consequence of inappropriate production of aldosterone and renin, which are typically and preferably selected from high plasma aldosterone levels, high plasma renin levels, hypertension, hypokalemic alkalosis that causes episodic weakness, paresthesia, transient paralysis, tetany, and peripheral edema. The term “secondary aldosteronism” also covers diseases or disorders that cause SA, such as reduced renal blood flow, obstructive renal artery disease (e.g., atheroma, stenosis), renal vasoconstriction, edematous disorders (e.g., heart failure, cirrhosis with ascites, nephrotic syndrome), a renin-producing tumor, a juxtaglomerular cell tumor, fibromuscular dysplasia, hyporeabsorption of sodium (as seen e.g. in Bartter and Gitelman syndromes), hypovolemia/hypotension. The term “secondary aldosteronism” covers also secondary hyperaldosteronism.

The term “subtyping” as used herein in the context of primary aldosteronism refers to the process by which it is identified if one or both adrenal glands are considered responsible for the uncontrolled overproduction of aldosterone. Subtyping involves using computed tomography (CT) scanning in combination with adrenal vein sampling (AVS) to distinguish between unilateral and bilateral disease. It is currently considered the most accurate method for diagnosis.

As used herein, the aldosterone-to-renin ratio (ARR) is understood to mean aldosterone concentration of the blood plasma divided by either the renin concentration or the renin activity in the blood. Aldosterone is measured as plasma (plasma is the liquid part of blood i.e., minus cells) aldosterone concentration (PAC). Renin is measured as plasma renin activity (PRA) or plasma renin concentration (PRC). To determine the ARR, a blood sample is drawn from the patient, and PAC divided by either PRA or PRC to determine the ARR. Without wising to be bound by theory, in preferred embodiments the ARR is used as a screening test to identify patients at risk of primary aldosteronism (PA).

Systolic blood pressure (SBP) as used herein is understood as the pressure generated by the heart on the arteries during ventricular contraction.

Diastolic blood pressure as used herein is understood as the arterial pressure between heart beats. Without wishing to be bound by theory, SBP is more closely correlated with cardiovascular events such as stroke and myocardial infarction. Both systolic and diastolic blood pressure are usually determined using a blood pressure cuff placed on the brachial artery (near the elbow). In primary aldosteronism (PA), blood pressure is increased due to the increased sodium and water retention in the body. Sodium and water are retained due to the excessive, uncontrolled production of aldosterone, a steroid hormone mainly responsible for sodium and subsequent water retention in the body. Hence, high blood pressure is considered a clinical marker of PA.

As used herein, “Office systolic blood pressure (oSBP)” is a blood pressure measurement taken in the doctor's office. The measurement of blood pressure indicates the blood pressure of the patient at the moment in time in the office.

As used herein, “Ambulatory systolic blood pressure (aSBP)” is a collection of blood pressure measurements taken over (generally) 24-hours. As used herein the blood pressure measurement was taken (in the case of study DP13C201) 3×/hour or every 20 minutes through the 24-hour period. The blood pressure monitor was attached to the subjects, allowing him/her to maintain their regular daily activities. Without wishing to be bound by theory, 24-hour blood pressure recordings are considered to be a more reflective, and hence more accurate, measure of a person's blood pressure as it is recorded continuously for 24-hours.

As used herein, the term “symptom” refers to any indication of an unusual state, disease or disorder and includes a departure from normal function or normal feeling which is either noticed by a subject, preferably patient, or objectively observable (effect or sign) reflecting the presence of the unusual state, disorder or disease.

As used herein, the term “day” or “daily” refers to either one calendar day or one 24-hour period, preferably the term “day” or “daily” refers to either one calendar day.

As used herein, the term “abnormal activity of aldosterone synthase” refers to an activity of aldosterone synthase which differs from the activity of the wild-type or native gene or protein, or which differs from the activity of the gene or protein in a healthy subject. The abnormal activity can be stronger or weaker than the normal activity.

As used herein, the term “normal” or the term “normalization” in the context of clinical parameters such as plasma aldosterone concentration (PAC), plasma renin activity (PRA), plasma renin concentration (PRC; also known as direct renin concentration (DRC)), plasma aldosterone-to-renin ratio (ARR), plasma potassium concentration, urine content of tetrahydroaldosterone (THA), ambulatory systolic blood pressure (aSBP), and office systolic blood pressure (oSBP) refers to cutoff values or thresholds or ranges defined by medical or clinical consensus publications on reference values as values characteristic of healthy individuals. Thus, a subject exhibiting a “normal” clinical parameter typically does not require additional clinical intervention to treat said parameter. The Endocrine Society Clinical Practice Guidelines suggest the following cutoff values to detect patients with primary aldosteronism (Funder et al., J Clin Endocrinol Metab 2016; 101:1889-1916), showing ARR cutoff values, depending on assay and based on whether PAC, PRA, and PRC (DRC) are measured in conventional or SI units:

| | PRA, | PRA, | DRC, | DRC, |
|---|---|---|---|---|
| | ng/mL/h | pmol/L/min | mU/L^{a} | ng/L^{a} |
| PAC (as ng/dL) | 20 | 1.6 | 2.4 | 3.8 |
| | 30^{b} | 2.5 | 3.7 | 5.7 |
| | 40 | 3.1 | 4.9 | 7.7 |
| PAC (as pmol/L) | 750^{b} | 60 | 91 | 144 |
| | 1000 | 80 | 122 | 192 |

^{a}Values shown are on the basis of a conversion factor of PRA (ng/mL/h) to DRC (mU/L) of 8.2. DRC assays are still in evolution, and in a recently introduced and already commonly used automated DRC assay, the conversion factor is 12 (see text). It should be noted that there is poor correlation between DRC and PRA in the range where PRA is <1 ng/mL/h, the domain of major interest in PA screening (221, 222).
^{b}The most commonly adopted cutoff values are 30 for PAC and PRA in conventional units (equivalent to 830 when PAC is in SI units) and 750 when PAC is expressed in SI units (equivalent to 27 in conventional units)

As used herein, a normal PAC is understood to be <15 ng/dL (<410 pmol/L) for adenoma PA patients and <10 ng/dL (<275 pmol/L) for bilateral PA patients.

As used herein, a normal PRA is understood to be >0.5 ng/mL/h and PRC with >5 ng/L with a sensitive assay that can detect PRA as low as 0.2-0.3 ng/mL/h or PRC as low as 2 mU/L for unilateral PA patients. As used herein, a normal PRA values for bilateral PA patients is understood as >1.0 ng/mL/h and PRC with >10 ng/L.

As used herein, a normal ARR is understood to be below 3.7 (ng/dL)/(mU/L) (i.e. PAC/DRC).

As used herein, a normal urinary aldosterone excretion is understood to be a below 12 μg/24 hour as measured by tetrahydroaldosterone content in a 24-hour urine samples.

As used herein, normal office or outpatient blood pressure is understood to be systolic blood pressure <140 mm Hg and diastolic blood pressure <90 mm Hg.

As used herein, normal ambulatory blood pressure is understood to be systolic blood pressure <135 mm Hg and diastolic blood pressure <85 mm Hg; preferably systolic blood pressure <130 mm Hg and diastolic blood pressure <80 mm Hg); and normal 24 h ambulatory blood pressure monitoring is understood to be systolic blood pressure <130 mm Hg and diastolic blood pressure <80 mmHg. Without wishing to be bound, these values are consistent with 2013 ESH/ESC guidelines for the management of arterial hypertension. J. Hypertens 2013; 31:1281-1357.

As used herein, normal potassium concentrations in the blood are understood to be potassium concentrations from 3.6 mmol/L to 5.2 mmol/L (Mayo Clinic). As used herein, levels below 3.6 mmol/L are understood to define states of hypokalemia whereas levels above 5.2 mmol/L are understood as states of hyperkalemia. As used herein, levels (i.e., potassium levels) below 3.0 mmol/L are understood to define states of severe hypokalemia.

As used herein, potassium-resistant hypokalemia refers to a state of hypokalemia, wherein said hypokalemia not treatable by administration of potassium, e.g., dietary potassium, to the subject with said potassium-resistant hypokalemia. In some embodiments, potassium-resistant hypokalemia is understood to mean that severe hypokalemia is unchanged (i.e., potassium concentration in the blood remains below 3.0 mmol/L), even after administration of potassium, e.g., dietary potassium, to said subject. In some embodiments, potassium-resistant hypokalemia is understood to mean that severe hypokalemia is changed to hypokalemia (i.e., potassium concentration in the blood is above 3.0 mmol/L but remains below 3.6 mmol/L) even after administration of potassium, e.g., dietary potassium, to said subject. In some embodiments, potassium-resistant hypokalemia is understood to mean that hypokalemia is unchanged (i.e., potassium concentration in the blood remains below 3.6 mmol/L) even after administration of potassium, e.g., dietary potassium, to said subject.

In some embodiments, the amount of potassium (e.g., dietary potassium) administered to said subject which is insufficient to treat said potassium-resistant hypokalemia is at least 60 mmol/day; preferably said amount is at least 70 mmol/day, more preferably at least 80 mmol/day.

As used herein, the term “inappropriate activity of aldosterone synthase” refers to the activity of aldosterone synthase of the wild-type or native gene or protein or to the activity of the gene or protein in a healthy subject, which is considered as appropriate in a healthy subject, but the same said activity is considered inappropriate for a diseased subject, i.e. said activity is too strong or too weak for a diseased subject.

The term “treating” and/or “treatment” as used herein refers to the management and care of a subject, preferably patient having a disease or disorder for which administration of one or more therapeutic compounds or compositions is indicated for the purpose of combating or alleviating symptoms and complications of those conditions. Treating includes administering one or more formulations of the present invention to prevent the onset of the symptoms or complications, alleviating the symptoms or complications, or eliminating the disease or disorder. As used herein, the term “treatment” (or “therapy” which are used interchangeably herein) refers to both therapeutic treatment and prophylactic or preventative measures. Preferably, “treatment” or “therapy” refers to therapeutic treatment.

The term “pharmaceutically acceptable excipient” as used herein includes any physiologically inert additive that is routinely used in pharmaceutical dosage forms. Pharmaceutically acceptable excipients are selected from the group comprising binders, diluents, carriers, lubricants, glidants, coating additives or combinations thereof. Additional suitable carriers for formulations of the active salts of the present invention can be found in Remington, The Science and Practice of Pharmacy, 2006, Lippincott Williams & Wilkins, Philadelphia.

As used herein, the term “patient” or “subject” is used to mean an animal, preferably a mammal, including a human affected by a “disease” or “disorder”. Examples of mammals include humans, dogs, cows, horses, pigs, sheep, goats, cats. In a particularly preferred embodiment said subject is a human. The terms “patient” and “human”, as used herein, are interchangeably used.

The term “administer” as used herein means to introduce or apply a therapeutic agent into or to the body of a subject, preferably patient in need thereof to treat a disease or condition.

The term “therapeutically effective amount” refers to a dosage of an active agent deemed to be effective to treat at least one sign or symptom of a disease or disorder or to provide a specific pharmacological response for which the active agent is administered. Amounts of an agent for administration may vary based upon the circumstances, e.g. diseased state of the subject, preferably patient, being treated, the dosage form, method of administration, subject factors, preferably patient factors and the like. A therapeutically effective amount of the active agent that is administered to a particular subject in a particular instance will not always be effective in treating the conditions described herein, even though such dosage is deemed to be a therapeutically effective amount by those of skill in the art.

The term “pharmaceutical composition” refers to a mixture containing at least one therapeutic agent to be administered to a subject, preferably a patient, in order to prevent or treat a particular disease or condition affecting the subject, preferably the patient.

As used herein, the term “for use” as used in “composition for use in treatment of a disease” shall disclose also the corresponding method of treatment and the corresponding use of a preparation for the manufacture of a medicament for the treatment of a disease.

As used herein, the term “chronic cardiovascular disease or disorder” is understood to mean a disease of the heart or blood vessels, wherein said disease is chronic. As used herein, the term “chronic” is understood as a disease or disorder that persists for at least eight weeks, preferably for at least three months, again more preferably for at least half a year, again more preferably for at least a one year, again more preferably for at least two years, again more preferably for at least 5 years, again more preferably for at least 10 years. In a very preferred embodiment a chronic disorder is a disorder lasting for the remainder of a patient's life.

As used herein, “with accompanying,” when used to describe two (or more) diseases or disorders, is understood to mean that a patient suffers from both diseases or disorders simultaneously. Thus, in preferred embodiments, a chronic cardiovascular disease or disorder with, e.g., accompanying hypokalemia is understood to refer to a patient or subject who exhibits both the chronic cardiovascular disease or disorder (e.g., primary aldosteronism) and hypokalemia at the same time. In some embodiments, said two diseases or disorders that “accompany” one another can also be associated with one another.

As used herein, the term “associated with” is understood to mean related to or connected with something else. In some embodiments wherein two (or more) diseases or disorders are associated with one another, one of the diseases or disorders causes and/or exacerbates (i.e., makes worse) the other disease or disorder. Thus, for example when a subject has a chronic cardiovascular disease associated with hypokalemia, the chronic cardiovascular disease or disorder (e.g., primary aldosteronism) is caused by or exacerbated by the hypokalemia. In some embodiments, the hypokalemia is caused by or exacerbated by the chronic cardiovascular disease or disorder (e.g., primary aldosteronism).

### Compounds of the Invention

As used herein, (R)-(+)-5-(p-cyanophenyl)-5,6,7,8-tetrahydroimidazo[1,5-a]pyridine is understood to mean a compound having the structure of formula (I) below, wherein said (R)-(+)-5-(p-cyanophenyl)-5,6,7,8-tetrahydroimidazo[1,5-a]pyridine has an enantiomeric excess (ee) of the (R) form of greater than or equal to 99%.

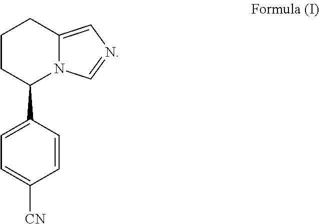 [SMILES: N#Cc1ccc([C@H]2CCCc3cncn32)cc1]

In some embodiments, the compound according to the invention is (R)-(+)-5-(p-cyanophenyl)-5,6,7,8-tetrahydroimidazo[1,5-a]pyridine or a pharmaceutically acceptable salt thereof. In some embodiments, said compound according to the invention is selected from the group consisting of (R)-(+)-5-(p-cyanophenyl)-5,6,7,8-tetrahydroimidazo[1,5-a]pyridine hydrochloride, (R)-(+)-5-(p-cyanophenyl)-5,6,7,8-tetrahydroimidazolium[1,5-a]pyridine dihydrogen phosphate, and (R)-(+)-5-(p-cyanophenyl)-5,6,7,8-tetrahydroimidazo[1,5-a]pyridine hydrogen phosphate. As used herein, the term “DP13” refers to a compound of Formula (I). In preferred embodiments, “DP13” refers to (R)-(+)-5-(p-cyanophenyl)-5,6,7,8-tetrahydroimidazolium[1,5-a]pyridine dihydrogen phosphate.

In preferred embodiments, said compound is (R)-(+)-5-(p-cyanophenyl)-5,6,7,8-tetrahydroimidazolium[1,5-a]pyridine dihydrogen phosphate.

In some embodiments, said compound is (R)-(+)-5-(p-cyanophenyl)-5,6,7,8-tetrahydroimidazo[1,5-a]pyridine or a pharmaceutically acceptable salt thereof, wherein said compound has an enantiomeric excess (ee) of the (R) form higher than or equal to about 99.5%. In preferred embodiments, said compound is (R)-(+)-5-(p-cyanophenyl)-5,6,7,8-tetrahydroimidazo[1,5-a]pyridine or a pharmaceutically acceptable salt thereof, wherein said compound has an enantiomeric excess (ee) of the (R) form higher than or equal to about 99.9%.

In some embodiments, said compound is selected from the group consisting of (R)-(+)-5-(p-cyanophenyl)-5,6,7,8-tetrahydroimidazo[1,5-a]pyridine hydrochloride, (R)-(+)-5-(p-cyanophenyl)-5,6,7,8-tetrahydroimidazolium[1,5-a]pyridine dihydrogen phosphate, and (R)-(+)-5-(p-cyanophenyl)-5,6,7,8-tetrahydroimidazo[1,5-a]pyridine hydrogen phosphate, wherein said compound has an enantiomeric excess (ee) of the (R) form higher than or equal to about 99.5%. In preferred embodiments, said compound is selected from the group consisting of (R)-(+)-5-(p-cyanophenyl)-5,6,7,8-tetrahydroimidazo[1,5-a]pyridine hydrochloride, (R)-(+)-5-(p-cyanophenyl)-5,6,7,8-tetrahydroimidazolium[1,5-a]pyridine dihydrogen phosphate, and (R)-(+)-5-(p-cyanophenyl)-5,6,7,8-tetrahydroimidazo[1,5-a]pyridine hydrogen phosphate, wherein said compound has an enantiomeric excess (ee) of the (R) form higher than or equal to about 99.9%.

In some embodiments, said compound is (R)-(+)-5-(p-cyanophenyl)-5,6,7,8-tetrahydroimidazolium[1,5-a]pyridine dihydrogen phosphate, wherein said compound has an enantiomeric excess (ee) of the (R) form higher than or equal to about 99.5%. In preferred embodiments, said compound is (R)-(+)-5-(p-cyanophenyl)-5,6,7,8-tetrahydroimidazolium[1,5-a]pyridine dihydrogen phosphate, wherein said compound has an enantiomeric excess (ee) of the (R) form higher than or equal to about 99.9%.

In another preferred embodiment, said pharmaceutically acceptable salt is crystalline. In another preferred embodiment, said pharmaceutically acceptable salt is anhydrous. In another preferred embodiment, said pharmaceutically acceptable salt is non-hygroscopic. In another preferred embodiment, said pharmaceutically acceptable salt is non-hygroscopic and anhydrous. In another preferred embodiment, said pharmaceutically acceptable salt is non-hygroscopic and crystalline. In another preferred embodiment, said pharmaceutically acceptable salt is non-hygroscopic, anhydrous and crystalline.

In one embodiment, the compound according to the invention is a crystalline form I of (R)-(+)-5-(p-cyanophenyl)-5,6,7,8-tetrahydroimidazolium[1,5-a]pyridine dihydrogen phosphate, said crystalline form I is characterized by an X-ray powder diffraction pattern comprising the following 20 values measured as described in the WO 2019/211394 (Bruker AXS D2 PHASER; Irradiation: CuKα (30 kV, 10 mA); scan range: 5 to 45° (2 theta value), sample rotation 5 rpm, 0.5 s/step, 0.010°/step, 3.0 mm detector slit): 19.504; 21.919 and 24.159. In one embodiment there is provided a crystalline form I of (R)-(+)-5-(p-cyanophenyl)-5,6,7,8-tetrahydroimidazolium[1,5-a]pyridine dihydrogen phosphate for use according to the invention, said crystalline form I is characterized by an X-ray powder diffraction pattern comprising the following 20 values measured: 19.504; 21.919 and 24.159, wherein each peak may vary by ±1 or preferably by ±0.5, or further preferably by ±0.2 degrees. In a preferred embodiment, said X-ray powder diffraction pattern further comprises the following 20 values: 16.003; 26.101; 27.168; 27.542 and 29.029. In a preferred embodiment, said X-ray powder diffraction pattern further comprises the following 20 values: 16.003; 26.101; 27.168; 27.542 and 29.029, wherein each peak may vary by ±1 or preferably by ±0.5, or further preferably by ±0.2 degrees. In a particularly preferred embodiment, there is provided a crystalline form I of (R)-(+)-5-(p-cyanophenyl)-5,6,7,8-tetrahydroimidazolium[1,5-a]pyridine dihydrogen phosphate for use according to the invention, said crystalline form I is characterized by an X-ray powder diffraction pattern comprising at least one, more preferably 2, 3, 4, 5, 6, 7, 8, 9, 10, 11, 12, 13, 14, 15, 16 or all of the following 20 values: 6.023129; 9.969034; 11.26224; 11.22848; 11.96566; 12.77761; 13.79347; 14.39314; 15.3394; 16.00317; 16.27337; 17.07502; 17.27593; 17.9904; 18.38238; 18.65471; 18.96096; 19.14281; 19.504; 20.01265; 20.58808; 20.43302; 20.72112; 21.12683; 21.91906; 22.59202; 24.44788; 24.15917; 24.48119; 25.70071; 26.10094; 26.58127; 27.16767; 27.54165; 27.71408; 28.27603; 28.09725; 28.54909; 29.02939; 29.71314; 30.07578; 30.68808; 30.92867; 31.6379; 32.27005; 32.79806; 33.20638; 33.23304; 33.65808; 34.41793; 34.35512; 35.02142; 35.06671; 35.68978; 35.93622; 36.50305; 36.56591; 36.92023; 37.14021; 39.60815; 37.89624 and 40.22464. In another particularly preferred embodiment, there is provided a crystalline form I of (R)-(+)-5-(p-cyanophenyl)-5,6,7,8-tetrahydroimidazolium[1,5-a]pyridine dihydrogen phosphate for use according to the invention, said crystalline form I is characterized by an X-ray powder diffraction pattern comprising at least one, more preferably 2, 3, 4, 5, 6, 7, 8, 9, 10, 11, 12, 13, 14, 15, 16 or all of the following 20 values: 6.023129; 9.969034; 11.26224; 11.22848; 11.96566; 12.77761; 13.79347; 14.39314; 15.3394; 16.00317; 16.27337; 17.07502; 17.27593; 17.9904; 18.38238; 18.65471; 18.96096; 19.14281; 19.504; 20.01265; 20.58808; 20.43302; 20.72112; 21.12683; 21.91906; 22.59202; 24.44788; 24.15917; 24.48119; 25.70071; 26.10094; 26.58127; 27.16767; 27.54165; 27.71408; 28.27603; 28.09725; 28.54909; 29.02939; 29.71314; 30.07578; 30.68808; 30.92867; 31.6379; 32.27005; 32.79806; 33.20638; 33.23304; 33.65808; 34.41793; 34.35512; 35.02142; 35.06671; 35.68978; 35.93622; 36.50305; 36.56591; 36.92023; 37.14021; 39.60815; 37.89624 and 40.22464, wherein each peak may vary by ±1 or preferably by ±0.5, or further preferably by ±0.2 degrees. In another particularly preferred embodiment, there is provided a crystalline form I of (R)-(+)-5-(p-cyanophenyl)-5,6,7,8-tetrahydroimidazolium[1,5-a]pyridine dihydrogen phosphate, characterized by an X-ray powder diffraction pattern comprising at least one, more preferably 2, 3, 4, 5, 6, 7, 8, 9, 10, 11, 12, 13, 14, 15, 16 or all of the following 20 values: 6.023129; 9.969034; 11.26224; 11.22848; 11.96566; 12.77761; 13.79347; 14.39314; 15.3394; 16.00317; 16.27337; 17.07502; 17.27593; 17.9904; 18.38238; 18.65471; 18.96096; 19.14281; 19.504; 20.01265; 20.58808; 20.43302; 20.72112; 21.12683; 21.91906; 22.59202; 24.44788; 24.15917; 24.48119; 25.70071; 26.10094; 26.58127; 27.16767; 27.54165; 27.71408; 28.27603; 28.09725; 28.54909; 29.02939; 29.71314; 30.07578; 30.68808; 30.92867; 31.6379; 32.27005; 32.79806; 33.20638; 33.23304; 33.65808; 34.41793; 34.35512; 35.02142; 35.06671; 35.68978; 35.93622; 36.50305; 36.56591; 36.92023; 37.14021; 39.60815; 37.89624 and 40.22464, wherein each peak may vary by ±0.5, or preferably by ±0.2 degrees. In one embodiment, the three largest peaks of crystalline form I in the XRPD diffractogram have a relative intensity of 1 to 0.85 to 0.55, especially of 1 to 0.9 to 0.6, more especially of 1 to 0.95 to 0.65, e.g. of 1 to 0.97 to 0.68 (obtainable by integration of each of the peaks in the XRPD diagrams). In a particular embodiment the largest peak is at a 2-theta (θ) value of about 21.919 and the second-largest peak is at a 2-theta (θ) value of about 19.504 and the third-largest peak is at a 2-theta (θ) value of about 24.159, respectively. In a further particular embodiment the largest peak is at a 2-theta (θ) value of about 21.919±0.5, or preferably by ±0.2 degrees, and the second-largest peak is at a 2-theta (θ) value of about 19.504±0.5, or preferably by ±0.2 degrees and the third-largest peak is at a 2-theta (θ) value of about 24.159±0.5, or preferably by ±0.2 degrees, respectively.

### Activity of the Inventive Compounds

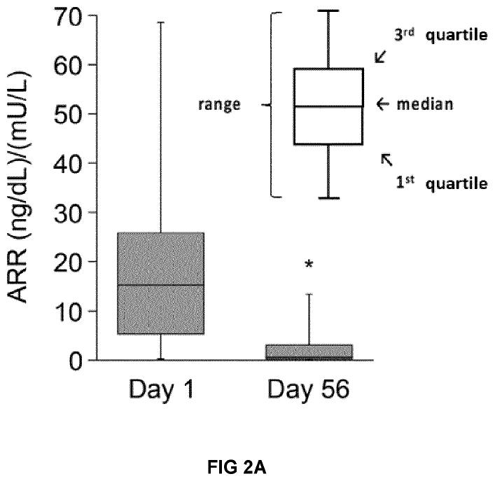

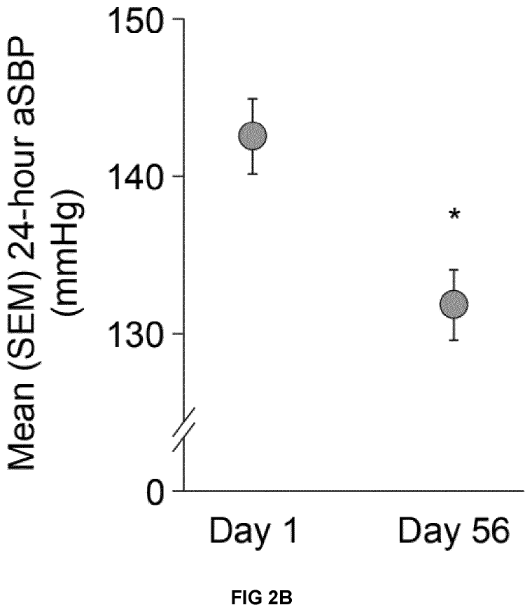

As shown below in Examples 1-5, the inventive compounds successfully corrected both biochemical (e.g., ARR) and clinical dysregulation (e.g., high blood pressure) caused by primary aldosteronism (i.e., in patients with primary aldosteronism). As shown in Example 2, Tables 1 and 2 and FIGS. 2A and 2B, treatment with dexfadrostat phosphate decreased median plasma ARR and mean 24-h aSBP at all doses tested (i.e., 4 mg, 8 mg, 12 mg) from baseline over a 56-day period.

Median ARR decreased in patients from 15.3 to 0.6 (ng/dL)/(mU/L) from baseline to day 56. Mean 24-h aSBP decreased by 10.7 mmHg from baseline to day 56.

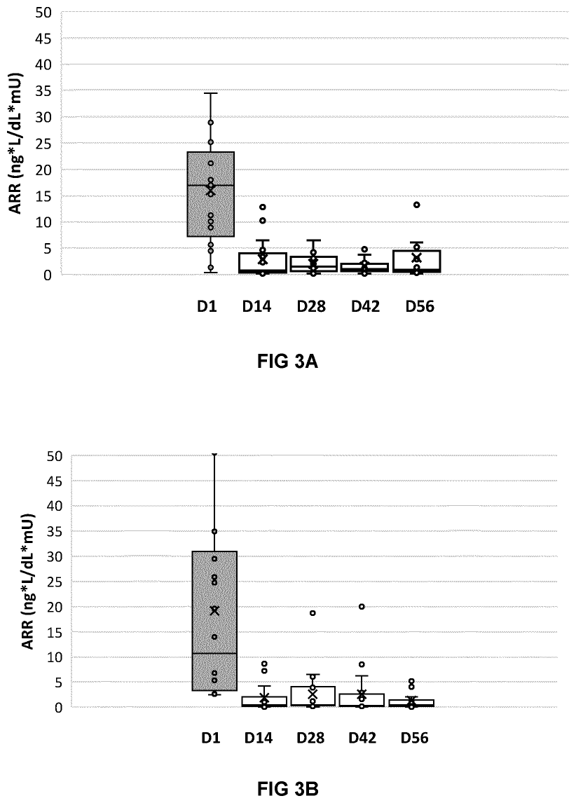

As shown in Example 2, Table 7, the inventive compounds successfully treated patients in patients with unilateral disease (e.g., unilateral adenoma or unilateral nodules). Specifically, treatment outcomes for patients (e.g., ARR) with unilateral PA were similar to the treatment outcomes for patients with bilateral PA or PA of undefined cause (idiopathic PA) in the respective dosage groups. Without wishing to be bound by theory, patients with unilateral primary aldosteronism (e.g., unilateral adenoma) are characterized by extreme aldosterone and potassium levels and typically require adrenal surgery for management. As shown in Example 2, Table 7, and FIGS. 3A and 3B, patients with unilateral PA were effectively treated with the inventive compounds (e.g., dexfadrostat phosphate) at the dosages tested, and patients with unilateral PA had responses similar to patients with bilateral PA or PA of undefined cause (idiopathic PA).

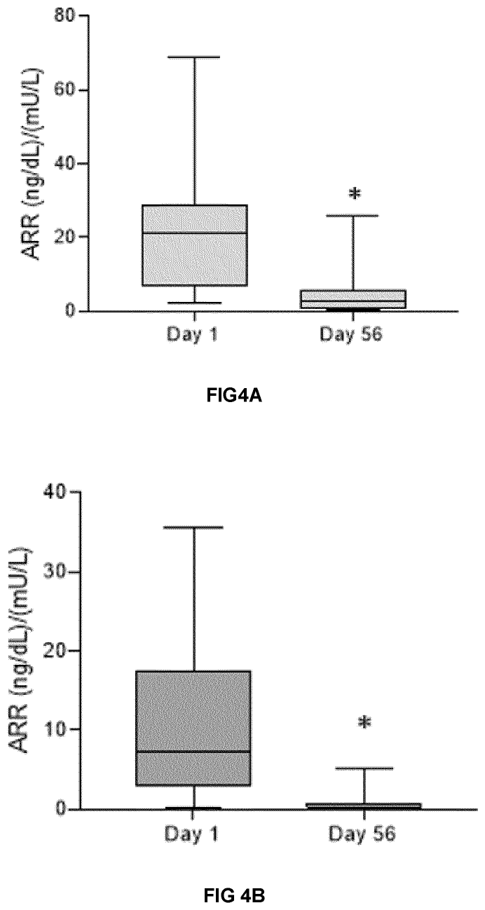

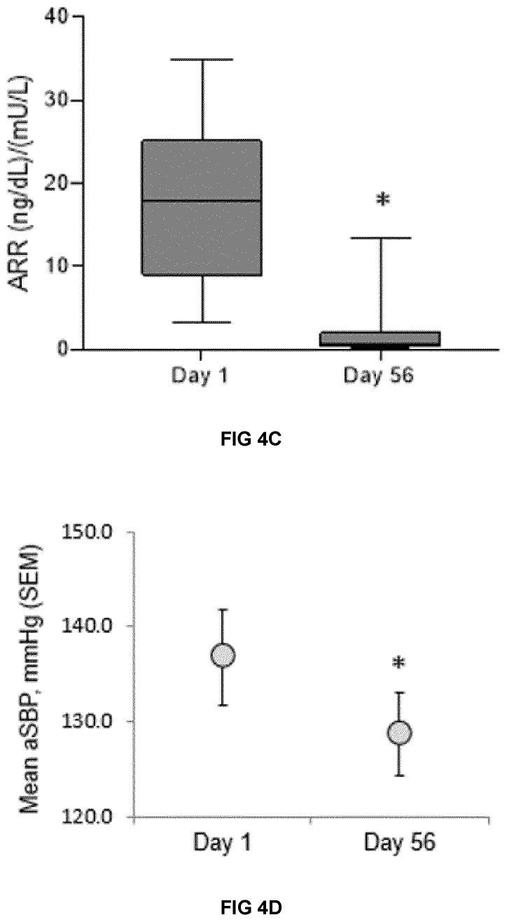

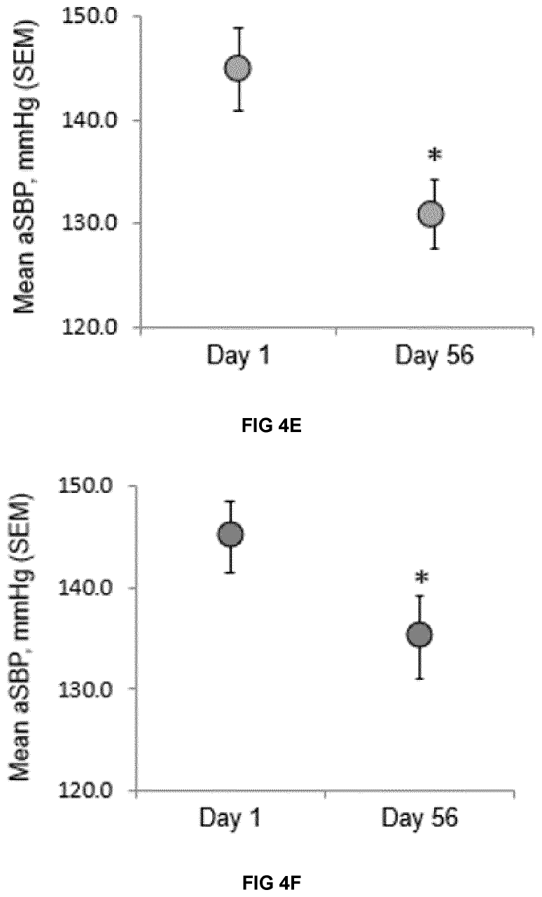

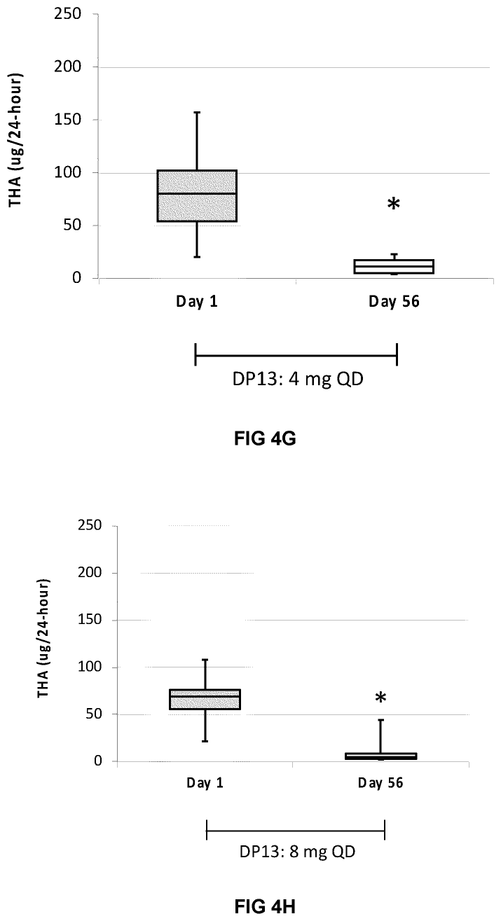

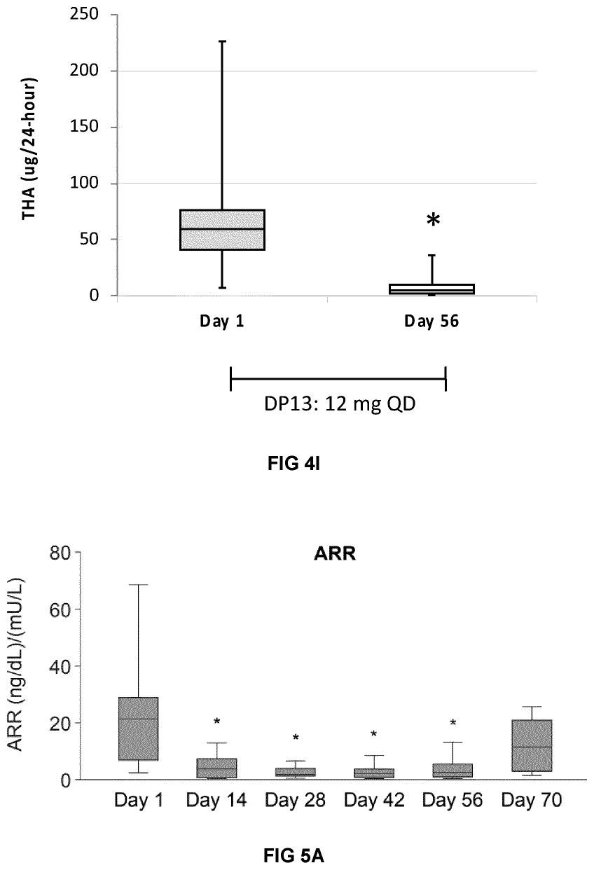

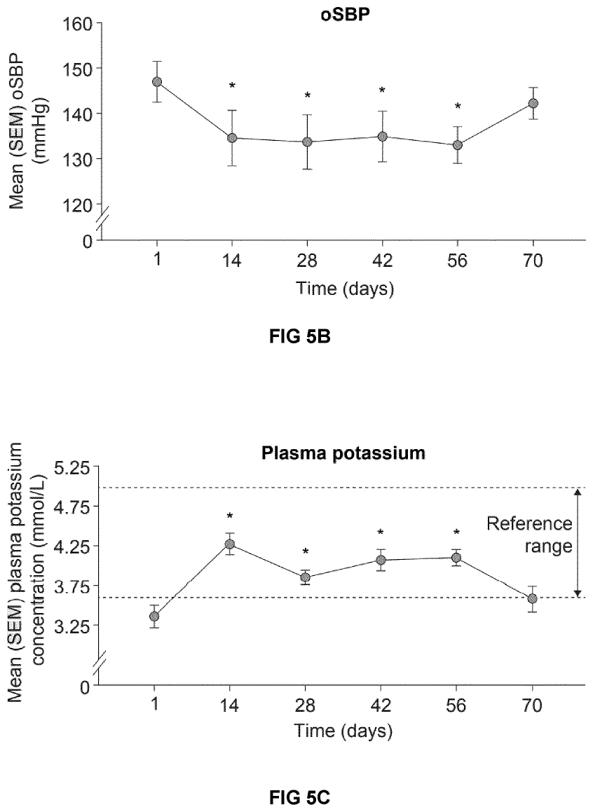

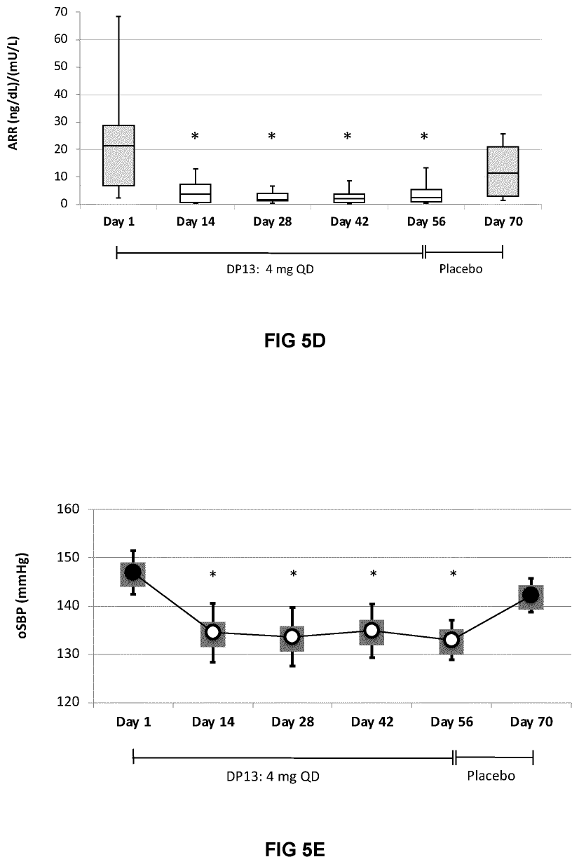

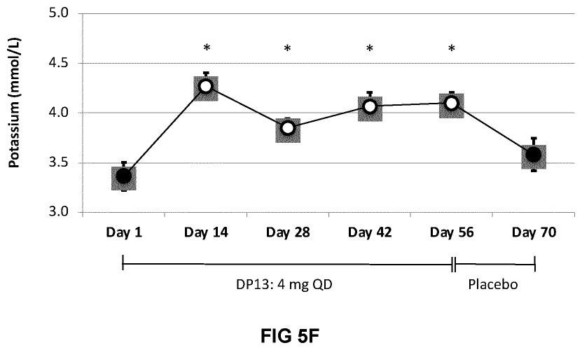

As shown in Example 3, Table 8, and FIGS. 4A, 4B and 4C, all three doses tested (i.e., 4 mg, 8 mg, and 12 mg) mediated significant reductions (P<0.0001) in ARR from baseline to day 56. The 4 mg dose was only slightly less efficacious than the 8 mg dose, and the 12 mg dose was not more potent than the 8 mg dose.

As shown in Example 4, Table 10, and FIGS. 4G, 4H and 4I, treatment with dexfadrostat phosphate at all three doses tested (i.e., 4 mg, 8 mg, and 12 mg) mediated significant reductions (P<0.0001) in tetrahydroaldosterone content, the main metabolite of aldosterone, in 24-hour urine samples collected at the end of active treatment compared to baseline samples.

Accordingly, in some embodiments, present invention provides for the use of the inventive compounds for the treatment of diseases characterized by abnormal aldosterone content in the urine.

As shown in Example 5, Tables 11-13 and FIGS. 5A-5F, treatment with dexfadrostat phosphate at all three doses tested mediated rapid and significant (p<0.0001) reductions in ARR, increase in plasma potassium concentration, and decrease in blood pressure from baseline to day 14, 28, 42, and 56 clinical visits. The ARR suppression, plasma potassium concentration, and decrease in blood pressure remained sustained over the entire active treatment period. Surprisingly, for all three clinical indicators tested, the 4 mg dose was almost as efficacious as the 8 mg dose and the 12 mg dose did not provide additional clinical benefit.

Accordingly, in some aspects, the present invention provides for the use of the inventive compounds for the rapid treatment of a disease or disorder (e.g., a disease or disorder wherein aldosterone overexposure contributes to the symptoms of said disease or disorder). Preferably said rapid treatment of said disease comprises treatment within 14 days. Without wishing to be bound by theory, current clinical responses to surgery and medical treatment can take 3-12 months to occur.

Moreover, in some aspects, the present invention provides for the use of the inventive compounds for sustained treatment over an extended treatment period (e.g., greater than eight weeks) without dose adjustment (e.g., at a constant once-daily dose of 4 mg or 8 mg). Without wishing to be bound by theory, current medical treatment requires bi-weekly dose adaptation.

As shown in the Examples below, the inventive compounds successfully treated hypokalemia, e.g., severe and/or potassium-resistant hypokalemia. The inventive compounds were able to treat hypokalemia in patients suffering from chronic cardiovascular diseases or disorders (e.g., primary aldosteronism). Without wishing to be bound by theory, in some embodiments the inventive compounds can also be used to treat hypokalemia e.g., severe and/or potassium-resistant hypokalemia in patients that do not have a cardiovascular disease or disorder, e.g., patients who do not have primary aldosteronism.

### Methods of Treatment

In one aspect, the present invention provides a composition for use in the treatment of a disease or disorder, wherein said composition is administered once daily to a subject in need thereof and wherein said composition comprises a compound which is (R)-(+)-5-(p-cyanophenyl)-5,6,7,8-tetrahydroimidazo[1,5-a]pyridine or a pharmaceutically acceptable salt thereof, wherein said compound has an enantiomeric excess (ee) of the (R) form higher than or equal to about 99%.

In one aspect, the present invention provides a composition for use in the treatment of a disease or disorder, wherein said composition is administered once daily to a subject in need thereof for at least eight weeks and wherein said composition comprises a compound which is (R)-(+)-5-(p-cyanophenyl)-5,6,7,8-tetrahydroimidazo[1,5-a]pyridine or a pharmaceutically acceptable salt thereof, wherein said compound has an enantiomeric excess (ee) of the (R) form higher than or equal to about 99%.

In a preferred embodiment, said subject in need of the composition of the invention are mammals. More preferably said subject is a human. Preferably, said humans include women of child bearing potential and pediatric patients. In a preferred embodiment, said humans are women of child bearing potential and pediatric patients. In some embodiments, said subject is a human.

In some embodiments, said composition is administered orally, preferably in the form of a tablet or capsule, yet more preferably in the form of a capsule.

In some embodiments, said composition is administered once daily to said subject at a dosage from about 1 mg to about 12 mg of said compound. In preferred embodiments, said composition is administered once daily to said subject at a dosage from about 1 mg to about 8 mg of said compound. In preferred embodiments, said composition is administered once daily to said subject at a dosage from 1 mg to 8 mg of said compound. In some embodiments, said composition is administered once daily to said subject at a dosage of about 4 mg or about 8 mg of said compound. In some embodiments, said composition is administered once daily to said subject at a dosage of 4 mg or 8 mg of said compound.

In some embodiments, said composition is administered once daily to said subject at a dosage of about 1 mg of said compound. In some embodiments, said composition is administered once daily to said subject at a dosage of about 2 mg of said compound. In some embodiments, said composition is administered once daily to said subject at a dosage of about 3 mg of said compound. In some embodiments, said composition is administered once daily to said subject at a dosage of about 4 mg of said compound. In some embodiments, said composition is administered once daily to said subject at a dosage of about 5 mg of said compound. In some embodiments, said composition is administered once daily to said subject at a dosage of about 6 mg of said compound. In some embodiments, said composition is administered once daily to said subject at a dosage of about 7 mg of said compound. In some embodiments, said composition is administered once daily to said subject at a dosage of about 8 mg of said compound.

In preferred embodiments, said composition is administered once daily to said subject at a dosage from about 1 mg to about 8 mg of said compound, wherein once daily administration does not require dose adjustment; preferably wherein said once daily administration does not require dose adjustment for at least eight weeks; preferably for at least six months; preferably for at least one year; preferably for at least five years; more preferably for at least 10 years; more preferably wherein said once daily administration never requires dose adjustment.

In preferred embodiments, said composition is administered once daily to said subject at a single daily dosage of about 4 mg for at least eight weeks; preferably for at least six months; preferably for at least one year; preferably for at least five years; more preferably for at least 10 years; more preferably wherein said once daily administration never requires dose adjustment. In preferred embodiments, said composition is administered once daily to said subject at a single daily dosage of about 8 mg for at least eight weeks; preferably for at least six months; preferably for at least one year; preferably for at least five years; more preferably for at least 10 years; more preferably wherein said once daily administration never requires dose adjustment.

In some embodiments, said subject requires rapid treatment of said disease or disorder. In one aspect, the present invention comprises eliciting a clinical response in a subject in need thereof, preferably within about 14 days of treatment. In some embodiments, said clinical response is selected from reduction in plasma aldosterone levels, normalization of plasma aldosterone levels, reduction of blood pressure, normalization of blood pressure, reduction of hypokalemia, normalization of potassium concentration, and reduction of urinary THA. Preferably said reduction in plasma aldosterone levels comprises achieving about <15 ng/dL (about <410 pmol/L) in a unilateral PA patients and about <10 ng/dL (about <275 pmol/L) for bilateral PA patients. Preferably said normalization in plasma aldosterone levels comprises achieving about <15 ng/dL (about <410 pmol/L) in a unilateral PA patients and about <10 ng/dL (about <275 pmol/L) for bilateral PA patients. Preferably said reduction of blood pressure comprises achieving systolic blood pressure about <135 mm Hg and diastolic blood pressure about <85 mm Hg; preferably systolic blood pressure about <130 mm Hg and diastolic blood pressure about <80 mm Hg) in a subject. Preferably said normalization of blood pressure comprises achieving systolic blood pressure about <135 mm Hg and diastolic blood pressure about <85 mm Hg; preferably systolic blood pressure about <130 mm Hg and diastolic blood pressure about <80 mm Hg) in a subject. Preferably said reduction of hypokalemia comprises achieving a plasma potassium concentration of about 3.6 mmol/L to about 5.2 mmol/L. Preferably said normalization of potassium concentration comprises achieving a plasma potassium concentration of about 3.6 mmol/L to about 5.2 mmol/L. Preferably said reduction of urinary THA comprises achieving urinary tetrahydroaldosterone excretion of less than about 12 g/24 hour. In one embodiment, the clinical response is reduction in ARR, preferably wherein said ARR is reduced to below about 5 (ng/dL)/(mU/L); preferably wherein said ARR is reduced to below about 4 (ng/dL)/(mU/L); preferably wherein said ARR is reduced to below about 3.7 (ng/dL)/(mU/L); preferably wherein said ARR is reduced to below about 3.5 (ng/dL)/(mU/L); preferably wherein said ARR is reduced to below about 3 (ng/dL)/(mU/L); preferably wherein said ARR is reduced to below about 2 (ng/dL)/(mU/L); preferably wherein said ARR is reduced to below about 1 (ng/dL)/(mU/L); preferably wherein said ARR is reduced to below about 0.55 (ng/dL)/(mU/L).

In some embodiments, said subject exhibits a clinical response within 14 days of treatment, wherein said clinical response is reduction in plasma aldosterone levels. Preferably said reduction in plasma aldosterone levels comprises achieving about <15 ng/dL (about <410 pmol/L) in a unilateral PA patients and about <10 ng/dL (about <275 pmol/L) for bilateral PA patients. In some embodiments, said subject exhibits a clinical response within 14 days of treatment, wherein said clinical response normalization of plasma aldosterone levels. Preferably said normalization in plasma aldosterone levels comprises achieving about <15 ng/dL (about <410 pmol/L) in a unilateral PA patients and about <10 ng/dL (about <275 pmol/L) for bilateral PA patients. In some embodiments, said subject exhibits a clinical response within 14 days of treatment, wherein said clinical response is reduction of blood pressure. Preferably said reduction of blood pressure comprises achieving systolic blood pressure about <135 mm Hg and diastolic blood pressure about <85 mm Hg; preferably systolic blood pressure about <130 mm Hg and diastolic blood pressure about <80 mm Hg) in a subject. In some embodiments, said subject exhibits a clinical response within 14 days of treatment, wherein said clinical response is normalization of blood pressure. Preferably said normalization of blood pressure comprises achieving systolic blood pressure about <135 mmHg and diastolic blood pressure about <85 mm Hg; preferably systolic blood pressure about <130 mm Hg and diastolic blood pressure about <80 mm Hg) in a subject. In some embodiments, said subject exhibits a clinical response within 14 days of treatment, wherein said clinical response is reduction of hypokalemia. Preferably said reduction of hypokalemia comprises achieving a plasma potassium concentration of about 3.6 mmol/L to about 5.2 mmol/L. In some embodiments, said subject exhibits a clinical response within 14 days of treatment, wherein said clinical response is normalization of potassium concentration. Preferably said normalization of potassium concentration comprises achieving a plasma potassium concentration of about 3.6 mmol/L to about 5.2 mmol/L. In some embodiments, said subject exhibits a clinical response within 14 days of treatment, wherein said clinical response is reduction of urinary THA. Preferably said reduction of urinary THA comprises achieving urinary tetrahydroaldosterone excretion of less than about 12 g/24 hour.

In some embodiments, said disease or disorder is a disease or disorder in which aldosterone overexposure contributes to the symptoms of the disease or disorder. In some embodiments said disease or disorder is selected from the group consisting of primary aldosteronism, secondary aldosteronism, heart failure, chronic renal failure, hypertension, restenosis, obesity, nephropathy, post-myocardial infarction, renal fibrosis, coronary heart disease, sodium retention, water retention, hypokalemia, hypomagnesaemia, hypertension, left ventricular hypertrophy, cardiac fibrosis, cardiovascular damage, suppression of plasma renin, kidney fibrosis, arrhythmias, nephropathy, edema, hypokalemia-caused muscle weakness, cardiac fibrillation and weakened cardiac muscle contraction;
preferably wherein said disease or disorder is selected from the group consisting of primary aldosteronism, secondary aldosteronism, heart failure, chronic renal failure, hypertension, restenosis, obesity, nephropathy, post-myocardial infarction, renal fibrosis, coronary heart disease, sodium retention, water retention, hypokalemia, hypomagnesaemia, hypertension, left ventricular hypertrophy, cardiac fibrosis, cardiovascular damage, suppression of plasma renin, kidney fibrosis, arrhythmias, nephropathy, and edema.

In preferred embodiments, said disease or disorder is selected from the group consisting of primary aldosteronism and secondary aldosteronism. In preferred embodiments, said disease or disorder is primary aldosteronism. In preferred embodiments, said disease or disorder is secondary aldosteronism.

In preferred embodiments, said disease or disorder characterized by abnormal aldosterone and/or tetrahydroaldosterone content in the urine. Preferably said disease or disorder characterized by abnormal aldosterone and/or tetrahydroaldosterone content in the urine is selected from primary aldosteronism, secondary aldosteronism, heart failure, chronic renal failure, hypertension, restenosis, obesity, nephropathy, post-myocardial infarction, renal fibrosis, coronary heart disease, sodium retention, water retention, hypokalemia, hypomagnesaemia, hypertension, left ventricular hypertrophy, cardiac fibrosis, cardiovascular damage, suppression of plasma renin, kidney fibrosis, arrhythmias, nephropathy, edema, hypokalemia-caused muscle weakness, cardiac fibrillation and weakened cardiac muscle contraction.

Preferably said disease or disorder characterized by abnormal aldosterone and/or tetrahydroaldosterone content in the urine is selected from the group consisting of primary aldosteronism, secondary aldosteronism, heart failure, chronic renal failure, hypertension, restenosis, obesity, nephropathy, post-myocardial infarction, renal fibrosis, coronary heart disease, sodium retention, water retention, hypokalemia, hypomagnesaemia, hypertension, left ventricular hypertrophy, cardiac fibrosis, cardiovascular damage, suppression of plasma renin, kidney fibrosis, arrhythmias, nephropathy, and edema.

In preferred embodiments, said disease or disorder characterized by abnormal aldosterone and/or tetrahydroaldosterone content in the urine is selected from primary aldosteronism, secondary aldosteronism, heart failure, renal failure, resistant hypertension, essential hypertension, nephropathy, edema, coronary heart disease, hypokalemia, and cardiac arrhythmias.

In preferred embodiments, said disease or disorder characterized by abnormal aldosterone and/or tetrahydroaldosterone content in the urine is selected from heart failure, renal failure, resistant hypertension, and essential hypertension.

In preferred embodiments, said disease or disorder is selected from the group consisting of primary aldosteronism and secondary aldosteronism. In preferred embodiments, said disease or disorder is primary aldosteronism. In preferred embodiments, said disease or disorder is secondary aldosteronism.

In some embodiments, said primary aldosteronism is unilateral or bilateral primary aldosteronism. In some embodiments, said unilateral or bilateral primary aldosteronism is caused by unilateral adenoma, bilateral adenoma, unilateral nodules, bilateral nodules, unilateral hyperplasia, bilateral hyperplasia, or normal-appearing adrenal glands.

In a very preferred embodiment, said disease or disorder is unilateral primary aldosteronism. In a preferred embodiment, said disease or disorder is unilateral primary aldosteronism caused by an aldosterone-producing adenoma (benign tumor), unilateral nodules, adrenal cancer, or hyperplasia, preferably unilateral hyperplasia. In a preferred embodiment, said disease or disorder is unilateral primary aldosteronism caused by an aldosterone-producing adenoma (benign tumor). In a preferred embodiment, said disease or disorder is unilateral primary aldosteronism caused by adrenal cancer. In a preferred embodiment, said disease or disorder is unilateral primary aldosteronism caused by hyperplasia, preferably unilateral hyperplasia. In a preferred embodiment, said disease or disorder is unilateral primary aldosteronism caused by unilateral nodules.

In a preferred embodiment, said primary aldosteronism is unilateral primary aldosteronism wherein said unilateral primary aldosteronism is un-detected or un-diagnosed.

In some embodiments, said primary aldosteronism is bilateral primary aldosteronism, e.g., bilateral adenoma.

In a preferred embodiment, said disease or disorder is essential hypertension and/or resistant hypertension, preferably wherein said disease or disorder is essential hypertension and/or resistant hypertension with accompanying hypokalemia. In a preferred embodiment, said disease or disorder is hypokalemia.

In one aspect, the present invention provides a composition for use in the treatment of a disease or disorder, wherein said composition is administered once daily to a subject in need thereof for at least eight weeks and wherein said composition comprises a compound which is (R)-(+)-5-(p-cyanophenyl)-5,6,7,8-tetrahydroimidazo[1,5-a]pyridine or a pharmaceutically acceptable salt thereof, wherein said compound has an enantiomeric excess (ee) of the (R) form higher than or equal to about 99%; wherein said disease or disorder is unilateral primary aldosteronism; preferably wherein said unilateral primary aldosteronism is caused by aldosterone-producing adenoma (benign tumor), unilateral nodules, adrenal cancer, or hyperplasia, preferably unilateral hyperplasia.

In one aspect, the present invention provides a composition for use in the treatment of a disease or disorder characterized by abnormal aldosterone content in the urine, wherein said composition is administered once daily to a subject in need thereof for at least eight weeks and wherein said composition comprises a compound which is (R)-(+)-5-(p-cyanophenyl)-5,6,7,8-tetrahydroimidazo[1,5-a]pyridine or a pharmaceutically acceptable salt thereof, wherein said compound has an enantiomeric excess (ee) of the (R) form higher than or equal to about 99%.

In one aspect, the present invention provides a composition for use in causing a clinical outcome in a subject in need thereof, wherein said composition is administered once daily to said subject for at least eight weeks and wherein said composition comprises a compound which is (R)-(+)-5-(p-cyanophenyl)-5,6,7,8-tetrahydroimidazo[1,5-a]pyridine or a pharmaceutically acceptable salt thereof, wherein said compound has an enantiomeric excess (ee) of the (R) form higher than or equal to about 99%. In some embodiments, said clinical outcome is selected from a reduction in plasma aldosterone levels, normalization of plasma aldosterone levels, reduction of blood pressure, normalization of blood pressure, reduction of hypokalemia, normalization of potassium plasma concentration, and reduction of urinary THA.

In some embodiments, the present invention provides a composition for use in reducing plasma aldosterone levels in a subject in need thereof, wherein said composition is administered once daily to said subject for at least eight weeks and wherein said composition comprises a compound which is (R)-(+)-5-(p-cyanophenyl)-5,6,7,8-tetrahydroimidazo[1,5-a]pyridine or a pharmaceutically acceptable salt thereof, wherein said compound has an enantiomeric excess (ee) of the (R) form higher than or equal to about 99%. Preferably said plasma aldosterone levels are reduced to a normal and/or non-pathological level.

In some embodiments, the present invention provides a composition for use in normalizing plasma aldosterone levels in a subject in need thereof, wherein said composition is administered once daily to said subject for at least eight weeks and wherein said composition comprises a compound which is (R)-(+)-5-(p-cyanophenyl)-5,6,7,8-tetrahydroimidazo[1,5-a]pyridine or a pharmaceutically acceptable salt thereof, wherein said compound has an enantiomeric excess (ee) of the (R) form higher than or equal to about 99%. Preferably said plasma aldosterone levels are reduced to a normal and/or non-pathological level.

In some embodiments, the present invention provides a composition for use in reducing blood pressure in a subject in need thereof, wherein said composition is administered once daily to said subject for at least eight weeks and wherein said composition comprises a compound which is (R)-(+)-5-(p-cyanophenyl)-5,6,7,8-tetrahydroimidazo[1,5-a]pyridine or a pharmaceutically acceptable salt thereof, wherein said compound has an enantiomeric excess (ee) of the (R) form higher than or equal to about 99%. Preferably said blood pressure is reduced to a normal and/or non-pathological level. In some embodiments said blood pressure is measured by aSBP. In some embodiments said blood pressure is measured by oSPB.

In some embodiments, the present invention provides a composition for use in normalizing blood pressure in a subject in need thereof, wherein said composition is administered once daily to said subject for at least eight weeks and wherein said composition comprises a compound which is (R)-(+)-5-(p-cyanophenyl)-5,6,7,8-tetrahydroimidazo[1,5-a]pyridine or a pharmaceutically acceptable salt thereof, wherein said compound has an enantiomeric excess (ee) of the (R) form higher than or equal to about 99%. Preferably said blood pressure is reduced to a normal and/or non-pathological level. In some embodiments said blood pressure is measured by aSBP. In some embodiments said blood pressure is measured by oSPB.

In some embodiments, the present invention provides a composition for use in reducing hypokalemia in a subject in need thereof, wherein said composition is administered once daily to said subject for at least eight weeks and wherein said composition comprises a compound which is (R)-(+)-5-(p-cyanophenyl)-5,6,7,8-tetrahydroimidazo[1,5-a]pyridine or a pharmaceutically acceptable salt thereof, wherein said compound has an enantiomeric excess (ee) of the (R) form higher than or equal to about 99%. Preferably said hypokalemia is reduced to a normal and/or non-pathological level.

In some embodiments, the present invention provides a composition for use in normalizing plasma potassium concentration in a subject in need thereof, wherein said composition is administered once daily to said subject for at least eight weeks and wherein said composition comprises a compound which is (R)-(+)-5-(p-cyanophenyl)-5,6,7,8-tetrahydroimidazo[1,5-a]pyridine or a pharmaceutically acceptable salt thereof, wherein said compound has an enantiomeric excess (ee) of the (R) form higher than or equal to about 99%. Preferably said plasma potassium concentration is increased to a normal and/or non-pathological level.

In some embodiments, the present invention provides a composition for use in reducing urinary THA levels in a subject in need thereof, wherein said composition is administered once daily to said subject for at least eight weeks and wherein said composition comprises a compound which is (R)-(+)-5-(p-cyanophenyl)-5,6,7,8-tetrahydroimidazo[1,5-a]pyridine or a pharmaceutically acceptable salt thereof, wherein said compound has an enantiomeric excess (ee) of the (R) form higher than or equal to about 99%. Preferably said urinary THA levels are reduced to a normal and/or non-pathological level.

In one aspect, the present invention provides a composition for use in the treatment of a disease or disorder, wherein said composition is administered once daily to a subject in need thereof for at least eight weeks and wherein said composition comprises a compound which is (R)-(+)-5-(p-cyanophenyl)-5,6,7,8-tetrahydroimidazo[1,5-a]pyridine or a pharmaceutically acceptable salt thereof, wherein said compound has an enantiomeric excess (ee) of the (R) form higher than or equal to about 99%; wherein said disease or disorder is selected from the group consisting of primary aldosteronism, secondary aldosteronism, heart failure, chronic renal failure, hypertension, restenosis, obesity, nephropathy, post-myocardial infarction, renal fibrosis, coronary heart disease, sodium retention, water retention, hypokalemia, hypomagnesaemia, hypertension, left ventricular hypertrophy, cardiac fibrosis, cardiovascular damage, suppression of plasma renin, kidney fibrosis, arrhythmias, nephropathy, edema, hypokalemia-caused muscle weakness, cardiac fibrillation and weakened cardiac muscle contraction.

In one aspect, the present invention provides a composition for use in the treatment of a disease or disorder, wherein said composition is administered once daily to a subject in need thereof for at least eight weeks and wherein said composition comprises a compound which is (R)-(+)-5-(p-cyanophenyl)-5,6,7,8-tetrahydroimidazo[1,5-a]pyridine or a pharmaceutically acceptable salt thereof, wherein said compound has an enantiomeric excess (ee) of the (R) form higher than or equal to about 99%; wherein said disease or disorder is selected from the group consisting of primary aldosteronism, and secondary aldosteronism.

In one aspect, the present invention provides a composition for use in the treatment of a disease or disorder, wherein said composition is administered once daily to a subject in need thereof for at least eight weeks and wherein said composition comprises a compound which is (R)-(+)-5-(p-cyanophenyl)-5,6,7,8-tetrahydroimidazo[1,5-a]pyridine or a pharmaceutically acceptable salt thereof, wherein said compound has an enantiomeric excess (ee) of the (R) form higher than or equal to about 99%; wherein said disease or disorder is primary aldosteronism.

In one aspect, the present invention provides a composition for use in the treatment of a disease or disorder, wherein said composition is administered once daily to a subject in need thereof for at least eight weeks and wherein said composition comprises a compound which is (R)-(+)-5-(p-cyanophenyl)-5,6,7,8-tetrahydroimidazo[1,5-a]pyridine or a pharmaceutically acceptable salt thereof, wherein said compound has an enantiomeric excess (ee) of the (R) form higher than or equal to about 99%; wherein said disease or disorder is secondary aldosteronism.

In one aspect, the present invention provides a composition for use in the treatment of a disease or disorder, wherein said composition is administered once daily to a subject in need thereof for at least eight weeks and wherein said composition comprises a compound which is (R)-(+)-5-(p-cyanophenyl)-5,6,7,8-tetrahydroimidazolium[1,5-a]pyridine dihydrogen phosphate, wherein said compound has an enantiomeric excess (ee) of the (R) form higher than or equal to about 99.5%, preferably higher than or equal to about 99.9%; wherein said disease or disorder is selected from the group consisting of primary aldosteronism, and secondary aldosteronism.

In one aspect, the present invention provides a composition for use in the treatment of a disease or disorder, wherein said composition is administered once daily at a dose of 4 mg to a subject in need thereof for at least eight weeks and wherein said composition comprises a compound which is (R)-(+)-5-(p-cyanophenyl)-5,6,7,8-tetrahydroimidazolium[1,5-a]pyridine dihydrogen phosphate, wherein said compound has an enantiomeric excess (ee) of the (R) form higher than or equal to about 99.5%, preferably higher than or equal to about 99.9%; wherein said disease or disorder is selected from the group consisting of primary aldosteronism, and secondary aldosteronism; preferably wherein said once daily 4 mg dose does not change for at least eight weeks.

In one aspect, the present invention provides a composition for use in the treatment of a disease or disorder, wherein said composition is administered once daily at a dose of 8 mg to a subject in need thereof for at least eight weeks and wherein said composition comprises a compound which is (R)-(+)-5-(p-cyanophenyl)-5,6,7,8-tetrahydroimidazolium[1,5-a]pyridine dihydrogen phosphate, wherein said compound has an enantiomeric excess (ee) of the (R) form higher than or equal to about 99.5%, preferably higher than or equal to about 99.9%; wherein said disease or disorder is selected from the group consisting of primary aldosteronism, and secondary aldosteronism; preferably wherein said once daily 8 mg dose does not change for at least eight weeks.

In one aspect, the present invention provides a composition for use in the treatment of a disease or disorder, wherein said composition is administered once daily at a dose of 4 mg to a subject in need thereof for at least eight weeks and wherein said composition comprises a compound which is (R)-(+)-5-(p-cyanophenyl)-5,6,7,8-tetrahydroimidazolium[1,5-a]pyridine dihydrogen phosphate, wherein said compound has an enantiomeric excess (ee) of the (R) form higher than or equal to about 99.5%, preferably higher than or equal to about 99.9%; wherein said disease or disorder is primary aldosteronism, preferably unilateral primary aldosteronism, more preferably wherein said unilateral primary aldosteronism is caused by an aldosterone-producing adenoma (benign tumor), unilateral nodules, adrenal cancer, or hyperplasia, preferably unilateral hyperplasia.

In one aspect, the present invention provides a composition for use in the treatment of a disease or disorder, wherein said composition is administered once daily at a dose of 8 mg to a subject in need thereof for at least eight weeks and wherein said composition comprises a compound which is (R)-(+)-5-(p-cyanophenyl)-5,6,7,8-tetrahydroimidazolium[1,5-a]pyridine dihydrogen phosphate, wherein said compound has an enantiomeric excess (ee) of the (R) form higher than or equal to about 99.5%, preferably higher than or equal to about 99.9%; wherein said disease or disorder is primary aldosteronism, preferably unilateral primary aldosteronism, more preferably wherein said unilateral primary aldosteronism is caused by an aldosterone-producing adenoma (benign tumor), unilateral nodules, adrenal cancer, or hyperplasia, preferably unilateral hyperplasia.

In one aspect, the present invention provides a composition for use in the treatment of a disease or disorder, wherein said composition is administered once daily to a subject in need thereof for at least eight weeks and wherein said composition comprises a compound which is (R)-(+)-5-(p-cyanophenyl)-5,6,7,8-tetrahydroimidazo[1,5-a]pyridine or a pharmaceutically acceptable salt thereof, wherein said compound has an enantiomeric excess (ee) of the (R) form higher than or equal to about 99%, wherein said disease or disorder is unilateral primary aldosteronism, wherein said composition is administered orally once daily to said subject at a dosage of about 4 mg or about 8 mg of said compound, and wherein said once-daily dose does not require dose adjustment. In preferred embodiments, said unilateral primary aldosteronism is caused by an aldosterone-producing adenoma (benign tumor), unilateral nodules, adrenal cancer, or hyperplasia, preferably unilateral hyperplasia. In preferred embodiments, said compound is administered at a dosage of about 4 mg. In preferred embodiments, said compound is administered at a dosage of about 8 mg. Preferably said compound is (R)-(+)-5-(p-cyanophenyl)-5,6,7,8-tetrahydroimidazolium[1,5-a]pyridine dihydrogen phosphate.

In one aspect, the present invention provides a composition for use in the treatment of unilateral primary aldosteronism, wherein said composition is administered orally once daily to a subject in need thereof for at least eight weeks at a dosage of about 4 mg, and wherein said composition comprises a compound which is (R)-(+)-5-(p-cyanophenyl)-5,6,7,8-tetrahydroimidazolium[1,5-a]pyridine dihydrogen phosphate, wherein said compound has an enantiomeric excess (ee) of the (R) form higher than or equal to about 99%. In preferred embodiments, said unilateral primary aldosteronism is caused by an aldosterone-producing adenoma (benign tumor), unilateral nodules, adrenal cancer, or hyperplasia, preferably unilateral hyperplasia.

In one aspect, the present invention provides a composition for use in the treatment of unilateral primary aldosteronism, wherein said composition is administered orally once daily to a subject in need thereof for at least eight weeks at a dosage of about 8 mg, and wherein said composition comprises a compound which is (R)-(+)-5-(p-cyanophenyl)-5,6,7,8-tetrahydroimidazolium[1,5-a]pyridine dihydrogen phosphate, wherein said compound has an enantiomeric excess (ee) of the (R) form higher than or equal to about 99%. In preferred embodiments, said unilateral primary aldosteronism is caused by an aldosterone-producing adenoma (benign tumor), unilateral nodules, adrenal cancer, or hyperplasia, preferably unilateral hyperplasia.

In one aspect, the present invention provides a composition for use in the treatment of a disease or disorder, wherein said disease or disorder is a chronic cardiovascular disease or disorder with accompanying hypokalemia, wherein said composition is administered orally once daily to a subject in need thereof, preferably for at least 8 weeks, wherein said composition comprises a compound which is (R)-(+)-5-(p-cyanophenyl)-5,6,7,8-tetrahydroimidazo[1,5-a]pyridine or a pharmaceutically acceptable salt thereof, wherein said compound has an enantiomeric excess (ee) of the (R) form higher than or equal to about 99%.

In one aspect, the present invention provides a composition for use in the treatment of a disease or disorder, wherein said disease or disorder is a chronic cardiovascular disease or disorder associated with hypokalemia, wherein said composition is administered orally once daily to a subject in need thereof, preferably for at least 8 weeks, wherein said composition comprises a compound which is (R)-(+)-5-(p-cyanophenyl)-5,6,7,8-tetrahydroimidazo[1,5-a]pyridine or a pharmaceutically acceptable salt thereof, wherein said compound has an enantiomeric excess (ee) of the (R) form higher than or equal to about 99%.

In some embodiments, the chronic cardiovascular disease or disorder with accompanying hypokalemia is selected from primary aldosteronism, secondary aldosteronism, heart failure, hypertension, restenosis, post-myocardial infarction, coronary heart disease, sodium retention, water retention, hypomagnesaemia, hypertension, left ventricular hypertrophy, cardiac fibrosis, cardiovascular damage, suppression of plasma renin, arrhythmias, edema, hypokalemia-caused muscle weakness, cardiac fibrillation and weakened cardiac muscle contraction; preferably wherein said chronic cardiovascular disease or disorder with accompanying hypokalemia is selected from the group consisting of primary aldosteronism, secondary aldosteronism, heart failure, hypertension, restenosis, post-myocardial infarction, coronary heart disease, hypertension, left ventricular hypertrophy, cardiac fibrosis, cardiovascular damage, arrhythmias, and edema.

In some embodiments, the chronic cardiovascular disease or disorder associated with hypokalemia is selected from primary aldosteronism, secondary aldosteronism, heart failure, hypertension, restenosis, post-myocardial infarction, coronary heart disease, sodium retention, water retention, hypomagnesaemia, hypertension, left ventricular hypertrophy, cardiac fibrosis, cardiovascular damage, suppression of plasma renin, arrhythmias, edema, hypokalemia-caused muscle weakness, cardiac fibrillation and weakened cardiac muscle contraction; preferably wherein said chronic cardiovascular disease or disorder associated with hypokalemia is selected from the group consisting of primary aldosteronism, secondary aldosteronism, heart failure, hypertension, restenosis, post-myocardial infarction, coronary heart disease, hypertension, left ventricular hypertrophy, cardiac fibrosis, cardiovascular damage, arrhythmias, and edema.

In some embodiments, the chronic cardiovascular disease or disorder with accompanying hypokalemia is primary aldosteronism with accompanying hypokalemia. In some embodiments, the chronic cardiovascular disease or disorder with accompanying hypokalemia is primary aldosteronism associated with hypokalemia.

In some embodiments, the chronic cardiovascular disease or disorder with accompanying hypokalemia is hyperaldosteronism with accompanying hypokalemia. In some embodiments, the chronic cardiovascular disease or disorder with accompanying hypokalemia is hyperaldosteronism associated with hypokalemia.

In some embodiments, the chronic cardiovascular disease or disorder with accompanying hypokalemia is primary aldosteronism with accompanying severe hypokalemia. In some embodiments, the chronic cardiovascular disease or disorder with accompanying hypokalemia is primary aldosteronism associated with severe hypokalemia.

In some embodiments, the chronic cardiovascular disease or disorder with accompanying hypokalemia is hyperaldosteronism with accompanying severe hypokalemia. In some embodiments, the chronic cardiovascular disease or disorder with accompanying hypokalemia is hyperaldosteronism associated with severe hypokalemia.

In some embodiments, the chronic cardiovascular disease or disorder with accompanying hypokalemia is primary aldosteronism with accompanying potassium-resistant hypokalemia. In some embodiments, the chronic cardiovascular disease or disorder with accompanying hypokalemia is primary aldosteronism associated with potassium-resistant hypokalemia.

In some embodiments, the chronic cardiovascular disease or disorder with accompanying hypokalemia is hyperaldosteronism with accompanying potassium-resistant hypokalemia. In some embodiments, the chronic cardiovascular disease or disorder with accompanying hypokalemia is hyperaldosteronism associated with potassium-resistant hypokalemia.

In some embodiments, said composition is administered once daily to said subject at a dosage from about 1 mg to about 12 mg of said compound. In some embodiments, said compound is administered at a dosage of about 4 mg. In some embodiments, said compound is administered at a dosage of about 8 mg.

In one aspect, the present invention provides a composition for use in the treatment of a disease or disorder, wherein said disease or disorder is hypokalemia, wherein said composition is administered orally once daily to a subject in need thereof, preferably for at least 8 weeks, wherein said composition comprises a compound which is (R)-(+)-5-(p-cyanophenyl)-5,6,7,8-tetrahydroimidazo[1,5-a]pyridine or a pharmaceutically acceptable salt thereof, wherein said compound has an enantiomeric excess (ee) of the (R) form higher than or equal to about 99%.

In some embodiments, the hypokalemia is severe hypokalemia. In some embodiments, the hypokalemia is potassium-resistant hypokalemia.

In some embodiments, the hypokalemia is hypokalemia with an accompanying chronic cardiovascular disease or disorder. In preferred embodiments, said chronic cardiovascular disease or disorder is selected from primary aldosteronism, secondary aldosteronism, heart failure, hypertension, restenosis, post-myocardial infarction, coronary heart disease, sodium retention, water retention, hypomagnesaemia, hypertension, left ventricular hypertrophy, cardiac fibrosis, cardiovascular damage, suppression of plasma renin, arrhythmias, edema, hypokalemia-caused muscle weakness, cardiac fibrillation and weakened cardiac muscle contraction; preferably wherein said chronic cardiovascular disease or disorder is selected from the group consisting of primary aldosteronism, secondary aldosteronism, heart failure, hypertension, restenosis, post-myocardial infarction, coronary heart disease, hypertension, left ventricular hypertrophy, cardiac fibrosis, cardiovascular damage, arrhythmias, and edema. In yet more preferred embodiments, said hypokalemia is hypokalemia with an accompanying chronic cardiovascular disease or disorder, wherein said chronic cardiovascular disease or disorder is primary aldosteronism. In some preferred embodiments, said hypokalemia is hypokalemia with an accompanying chronic cardiovascular disease or disorder, wherein said chronic cardiovascular disease or disorder is hyperaldosteronism.

In some embodiments, the severe hypokalemia is severe hypokalemia with an accompanying chronic cardiovascular disease or disorder. In preferred embodiments, said chronic cardiovascular disease or disorder is selected from primary aldosteronism, secondary aldosteronism, heart failure, hypertension, restenosis, post-myocardial infarction, coronary heart disease, sodium retention, water retention, hypomagnesaemia, hypertension, left ventricular hypertrophy, cardiac fibrosis, cardiovascular damage, suppression of plasma renin, arrhythmias, edema, hypokalemia-caused muscle weakness, cardiac fibrillation and weakened cardiac muscle contraction; preferably wherein said chronic cardiovascular disease or disorder is selected from the group consisting of primary aldosteronism, secondary aldosteronism, heart failure, hypertension, restenosis, post-myocardial infarction, coronary heart disease, hypertension, left ventricular hypertrophy, cardiac fibrosis, cardiovascular damage, arrhythmias, and edema. In yet more preferred embodiments, said severe hypokalemia is severe hypokalemia with an accompanying chronic cardiovascular disease or disorder, wherein said chronic cardiovascular disease or disorder is primary aldosteronism. In some preferred embodiments, said severe hypokalemia is severe hypokalemia with an accompanying chronic cardiovascular disease or disorder, wherein said chronic cardiovascular disease or disorder is hyperaldosteronism.

In some embodiments, the potassium-resistant hypokalemia is potassium-resistant hypokalemia with an accompanying chronic cardiovascular disease or disorder. In preferred embodiments, said chronic cardiovascular disease or disorder is selected from primary aldosteronism, secondary aldosteronism, heart failure, hypertension, restenosis, post-myocardial infarction, coronary heart disease, sodium retention, water retention, hypomagnesaemia, hypertension, left ventricular hypertrophy, cardiac fibrosis, cardiovascular damage, suppression of plasma renin, arrhythmias, edema, hypokalemia-caused muscle weakness, cardiac fibrillation and weakened cardiac muscle contraction; preferably wherein said chronic cardiovascular disease or disorder is selected from the group consisting of primary aldosteronism, secondary aldosteronism, heart failure, hypertension, restenosis, post-myocardial infarction, coronary heart disease, hypertension, left ventricular hypertrophy, cardiac fibrosis, cardiovascular damage, arrhythmias, and edema. In yet more preferred embodiments, said potassium-resistant hypokalemia is potassium-resistant hypokalemia with an accompanying chronic cardiovascular disease or disorder, wherein said chronic cardiovascular disease or disorder is primary aldosteronism. In some preferred embodiments, said potassium-resistant hypokalemia is potassium-resistant hypokalemia with an accompanying chronic cardiovascular disease or disorder, wherein said chronic cardiovascular disease or disorder is hyperaldosteronism.

In some embodiments, the severe hypokalemia is severe hypokalemia associated with a chronic cardiovascular disease or disorder. In preferred embodiments, said chronic cardiovascular disease or disorder is selected from primary aldosteronism, secondary aldosteronism, heart failure, hypertension, restenosis, post-myocardial infarction, coronary heart disease, sodium retention, water retention, hypomagnesaemia, hypertension, left ventricular hypertrophy, cardiac fibrosis, cardiovascular damage, suppression of plasma renin, arrhythmias, edema, hypokalemia-caused muscle weakness, cardiac fibrillation and weakened cardiac muscle contraction; preferably wherein said chronic cardiovascular disease or disorder is selected from the group consisting of primary aldosteronism, secondary aldosteronism, heart failure, hypertension, restenosis, post-myocardial infarction, coronary heart disease, hypertension, left ventricular hypertrophy, cardiac fibrosis, cardiovascular damage, arrhythmias, and edema. In yet more preferred embodiments, said severe hypokalemia is severe hypokalemia associated with a chronic cardiovascular disease or disorder, wherein said chronic cardiovascular disease or disorder is primary aldosteronism. In some preferred embodiments, said severe hypokalemia is severe hypokalemia associated with a chronic cardiovascular disease or disorder, wherein said chronic cardiovascular disease or disorder is hyperaldosteronism.

In some embodiments, the potassium-resistant hypokalemia is potassium-resistant hypokalemia associated with a chronic cardiovascular disease or disorder. In preferred embodiments, said chronic cardiovascular disease or disorder is selected from primary aldosteronism, secondary aldosteronism, heart failure, hypertension, restenosis, post-myocardial infarction, coronary heart disease, sodium retention, water retention, hypomagnesaemia, hypertension, left ventricular hypertrophy, cardiac fibrosis, cardiovascular damage, suppression of plasma renin, arrhythmias, edema, hypokalemia-caused muscle weakness, cardiac fibrillation and weakened cardiac muscle contraction; preferably wherein said chronic cardiovascular disease or disorder is selected from the group consisting of primary aldosteronism, secondary aldosteronism, heart failure, hypertension, restenosis, post-myocardial infarction, coronary heart disease, hypertension, left ventricular hypertrophy, cardiac fibrosis, cardiovascular damage, arrhythmias, and edema. In yet more preferred embodiments, said potassium-resistant hypokalemia is potassium-resistant hypokalemia associated with a chronic cardiovascular disease or disorder, wherein said chronic cardiovascular disease or disorder is primary aldosteronism. In some preferred embodiments, said potassium-resistant hypokalemia is potassium-resistant hypokalemia associated with a chronic cardiovascular disease or disorder, wherein said chronic cardiovascular disease or disorder is hyperaldosteronism.

In some embodiments, said composition is administered once daily to said subject at a dosage from about 1 mg to about 12 mg of said compound. In some embodiments, said compound is administered at a dosage of about 4 mg. In some embodiments, said compound is administered at a dosage of about 8 mg.

In one aspect, the present invention provides a composition for use in the treatment of a disease or disorder, wherein said disease or disorder is hypokalemia (e.g., severe hypokalemia and/or potassium-resistant hypokalemia), wherein said composition is administered orally once daily to a subject in need thereof, preferably for at least 8 weeks, wherein said composition comprises a compound which is (R)-(+)-5-(p-cyanophenyl)-5,6,7,8-tetrahydroimidazo[1,5-a]pyridine or a pharmaceutically acceptable salt thereof, wherein said compound has an enantiomeric excess (ee) of the (R) form higher than or equal to about 99%, and wherein said hypokalemia is not accompanied by or associated with an additional disease or disorder. In some embodiments, said composition is administered once daily to said subject at a dosage from about 1 mg to about 12 mg of said compound. In some embodiments, said compound is administered at a dosage of about 4 mg. In some embodiments, said compound is administered at a dosage of about 8 mg.

In a preferred embodiment, said composition for use according to the invention further comprises at least one pharmaceutically acceptable excipient. In one embodiment, said pharmaceutical composition is formulated for oral administration. Said oral formulation is preferably solid. More preferably said oral formulation is selected from the group consisting of a tablet, pill, dispersible granule, cachet, capsule, powder, lozenge, suppository, and retention enemas.

In one embodiment, said pharmaceutical composition is an oral unit dose form. In a further embodiment, said pharmaceutical composition is an oral solid unit dose forms e.g. tablets and capsules etc.

In a preferred embodiment, said composition for use according to the invention is administered once daily orally. In an also preferred embodiment, said composition for use according to the invention is formulated for oral administration (oral formulation). Said oral formulation is preferably solid. More preferably said oral formulation is selected from the group consisting of a tablet, pill, dispersible granule, cachet, capsule, powder, lozenge, suppository, and retention enema, most preferably of a capsule.

In a preferred embodiment, said composition for use according to the invention is administered for a period of n consecutive days, wherein n is preferably >1. In a preferred embodiment, said composition for use according to the invention is administered once daily consecutively over a period of time. In a very preferred embodiment, said composition for use according to the invention is administered chronically. In another preferred embodiment, said composition for use according to the invention is administered for at least one month, preferably for at least two months, more preferably for at least three months, again more preferably for at least half a year, again more preferably for at least a one year, again more preferably for at least two years, again more preferably for at least 5 years, again more preferably for at least 10 years. In a very preferred embodiment said composition for use according to the invention is a life-time treatment.

### EXAMPLES

### Equipment, Materials and Methods

(R)-(+)-5-(p-cyanophenyl)-5,6,7,8-tetrahydroimidazolium[1,5-a]pyridine dihydrogen phosphate (also known as dexfadrostat phosphate) was prepared according to the teachings of PCT/EP2017/077511 (see WO 2018/078049), Example 3 in >99.9% ee.

### Example 1

### A Double-Blind, Randomized, Phase 2 Trial in Patients with Primary Aldosteronism to Investigate the Efficacy of Dexfadrostat Phosphate to Correct Biochemical (High Plasma ARR) and Clinical (High Blood Pressure) Disease Parameters

### Study Design

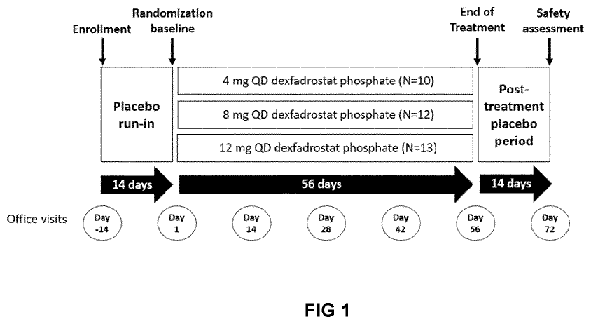

A double-blind, randomized, Phase 2 trial was conducted between November 2019 and June 2022 at 11 centres in Italy, Switzerland and the Netherlands (NCT04007406). After a 14-day single-blind, placebo run-in period, patients were randomized 1:1:1 to receive oral dexfadrostat phosphate capsules once-daily for 56 days followed by a single-blind, 14-day placebo withdrawal period (FIG. 1). Patients requiring fixed-dose antihypertensive therapy continued treatment throughout the study; steroid therapies, β-adrenergic and renin-angiotensin-aldosterone system blockers were not permitted. At 14-day intervals, office blood pressures were recorded and blood samples were taken for measurement of steroid and peptide hormones and electrolytes at a central laboratory. At baseline (day 1) and at end of active treatment (day 56), ambulatory blood pressure was recorded over 24-hour and a 24-hour urine samples were collected.

### Office Blood Pressure Measurement

Blood pressure and pulse rate were measured at each visit after a >15-minute rest in sitting position. Blood pressure and pulse rate were measured using a locally calibrated and validated automated oscillometric device (e.g., Omron Healthcare). An adapted wrap cuff was placed on the dominant arm. Three repeat measurements with a 10-minute interval were recorded; the average of the 2^{nd} and 3^{rd} measurements were study relevant.

### Ambulatory Blood Pressure Measurement

Twenty-four-hour ambulatory blood pressure monitoring (ABPM) included mean day- and night-time systolic and diastolic blood pressure recordings (measured every 20 minutes) along with heart rate measurements on day −1 prior to administration of the first dexfadrostat phosphate dose and on day 55 after the administration of the second-to-last dexfadrostat phosphate dose. A centrally calibrated and validated device (Spacelab Healthcare) was used; the device was fitted at the hospital outpatient department by medically qualified staff on day −1 and day 55 prior to supervised drug intake by the patient. The light digital monitor with convenient carry pouches takes the blood pressure reading at regular intervals by inflating and subsequently deflating customized cuffs placed around the non-dominant upper arm for 24-hour recording of about 70 readings. Patients followed their regular routines yet refrained from exercising or taking a bath or shower. In addition, to assure proper measurements, the patient was asked to sit down prior to reading, keep the cuff at the same level as the heart and rest the arm steady. The patient was also asked to keep a diary on activities, meals and sleep to document what he/she was doing just before the reading was taken. At the end of the 24-hour period, the patient was able to remove the monitor and cuff, shower or bathe and return the device to the Investigator at the clinic. The machine stored all readings as source data that were uploaded in the eCRF.

### Blood Sample Analysis

Plasma aldosterone and plasma renin concentration were measured centrally under GCP regulations using the Liaison® chemiluminescent immunoassay kit supplied by DiaSorin S.p.A. Italy. The system used specific monoclonal antibodies to prevent cross reactions with other major potential reactants in the serum samples. The monoclonal antibodies directed against specific epitopes of the renin molecule allowed accurate and sensitive quantification. The assays were performed according to the instructions provided by the manufacturer. The aldosterone assay had a wide measuring range of 0.97-100 ng/ml and was designed for assessing aldosterone to diagnose and evaluate treatments for PA. The analytical sensitivity was <0.97 ng/ml. The renin assay had a wide measuring range up to 500 IU/mL and was designed for assessing renin to diagnose and evaluate treatments for PA. The analytical sensitivity was <0.53 IU/mL.

### Urine Sample Analysis

The urinary content of tetrahydroaldosterone (THA) was measured centrally by gas chromatography-coupled mass spectrometry (GC-MS).

### Patient Eligibility

Key inclusion criteria for patients included in the study were: (i) aged 18 to 65 years; (ii) diagnosis of PA within 1 year of enrolment using Endocrine Society diagnostic criteria; (iii) excessive and autonomous aldosterone production confirmed by ARR before and after suppression test; (iv) and office systolic blood pressure (oSBP) greater than 145 mmHg and less than 190 mmHg.

Specifically, PA diagnosis was defined by plasma levels of aldosterone, plasma renin activity (PRA) or plasma renin concentration (PRC) prior or after a 4-hour IV saline infusion or a 2-hour captopril suppression test using the following protocol-specified cut-off values:
ARR of ≥40 with plasma aldosterone concentration (PAC) ≥15 ng/dL and plasma renin activity (PRA)<1.0 ng/mL/h. A pre-screening ARR of ≥3.7 resulted by using plasma renin concentration (PRC)<15 mU/L instead of PRA as denominator.
A IV saline loading test (2 litres of 0.9% saline infused over 4 hours) with resulting PAC >7.0 ng/dL after infusion. For patients at risk for volume expansion, a captopril test (50 mg captopril peroral) was used instead with resulting ARR >30 and PAC >11 ng/dL (respectively ARR >2.4 using PRC in mU/L instead of PRA as denominator).
   The protocol did not require patients to be further qualified by CT adrenal imaging or adrenal vein sampling.

Key exclusion criteria were: (i) oSBP >190 mmHg; (ii) hyperkalaemia; and (iii) prolonged QT interval.

For Endocrine Society diagnostic criteria, see Funder, J. W. et al., J Clin. Endocrinol. Metab., 2016; 101:1889-1916.

### Endpoints

Co-primary endpoints were change from baseline to day 56 in: (i) median ARR (as measured by aldosterone and renin concentrations, for all dose groups combined; and (ii) mean 24-hour ambulatory systolic blood pressure (aSBP) (for all dose groups combined). Secondary and exploratory endpoints included: (i) change from baseline to day 56 in aSBP stratified by dose; (ii) change from baseline to days 14, 28, 42, 56 and 70 in plasma ARR, oSBP, and potassium stratified by dose; (iii) change in 24-hour urinary tetrahydroalosterone excretion from baseline to the end of treatment on day 56; and (iv) treatment-emergent adverse events (TEAE) and serious adverse events reported between baseline and day 70.

### Statistical Analysis

For all efficacy endpoints, changes from baseline were calculated from linear models and accompanied by 95% confidence intervals and p values. Log-transformed values were used for changes in ARR. The full analysis set (FAS) and safety set were defined as all randomized patients who received at least one dose of study drug.

### Demographics and Baseline Characteristics

One patient discontinued during the placebo-run-in period owing to COVID-19 hospital restrictions and was not included in the efficacy or safety analyses. The full analysis set (FAS) and safety set both comprised the remaining 35 patients of whom 74.3% were male and 91.4% were white (Table 1). At baseline (day 1), the median (range) age was 53.0 (30-64) years; mean (SD) oSBP was 147.7 (11.8) mmHg; mean (SD) plasma potassium concentration was 3.5 (0.4) mmol/L; and 31% of patients had hypokalemia. Cardiac, vascular, and renal or urinary disorders were present in 25.7%, 34.3% and 8.6% of patients, respectively; one patient had a previous adrenalectomy (see Table 2).

TABLE 1
Patient Demographics and Characteristics at Baseline

| Demographic/characteristic (N = 35) | | |
|---|---|---|
| Mean age, years (SD) | 51.9 | (8.7) |
| Median age, years (range) | 53.0 | (30-64) |
| Female, n (%) | 9 | (25.7) |
| Child-bearing potential | 5 | (55.6%) |
| Male, n (%) | 26 | (74.3) |
| Race, n (%) | | |
| Asian | 1 | (2.9) |
| Black or African American | 2 | (5.7) |
| White | 32 | (91.4) |
| Mean oSBP, mmHg (SD) | 147.7 | (11.8) |
| Mean serum potassium, mmol/L (SD) | 3.6 | (0.4) |
| Mean oDBP, mmHG (SD) | 92.4 | (9.9) |
| Mean GFR, mL/min/1.73 m² (SD) | 95.1 | (18.3) |
| Mean Weight, kg (SD) | 84.1 | (12.9) |
| Mean BMI, kg/m² (SD) | 28.2 | (3.6) |

| | |
|---|---|
| PA diagnosis de novo (within 10 weeks) | 25 |
| PA diagnosis within one year | 10 |

TABLE 2
Patient Concomitant Diseases and Previous Procedures at Baseline

| MedDRA System Organ Class / Preferred Term | Total (N = 35) n (%) | |
|---|---|---|
| Metabolism and nutrition disorders | 23 | (65.7) |
| Hypokalemia | 11 | (31.4) |
| Obesity | 7 | (20.0) |
| Type 2 diabetes mellitus | 5 | (14.3) |
| Vascular disorders | 12 | (34.3) |
| Hypertension | 11 | (31.4) |
| Cardiac disorders | 9 | (25.7) |
| Hypertensive cardiomyopathy | 2 | (5.7) |
| Left ventricular hypertrophy | 2 | (5.7) |
| Congenital, familial and genetic disorders | 5 | (14.3) |
| Musculoskeletal and connective tissue disorders | 4 | (11.4) |
| Respiratory, thoracic and mediastinal disorders | 4 | (11.4) |
| Obstructive sleep apnea | 4 | (11.4) |
| Previous surgical and medical procedures | 12 | (34.3) |
| Adrenalectomy | 1 | (2.9) |

TABLE 3
Patient Hypertension Control Therapy at Baseline

| | Total (N = 35); n (%) | |
|---|---|---|
| Number of patients with at least one fixed | 32 | (91.43) |
| hypertension control therapy | | |
| Hypertension control therapy / Doses | | |
| Amlodipine (mg QD) | 17; | (48.57) |
| 2.5 | 1; | (2.86) |
| 5 | 5; | (14.29) |
| 10 | 11; | (31.43) |
| Diltiazem (mg QD) | 1; | (2.86) |
| 300 | 1; | (2.86) |
| Doxazosin (mg QD) | 24; | (68.57) |
| 1 | 1; | (2.86) |
| 2 | 5; | (14.29) |
| 4 | 11 | (31.43) |
| 8 | 7; | (20.00) |
| Verapamil (mg BID) | 5; | (14.29) |
| 40 | 1; | (2.86) |
| 80 | 1; | (2.86) |
| 120 | 3; | (8.57) |

### Primary Endpoints

The primary endpoints of the study assessed whether daily dexfadrostat phosphate treatment for 8 weeks corrected the biochemical (ARR) and clinical dysregulation (high blood pressure) caused by primary aldosteronism. Dexfadrostat phosphate treatment decreased median plasma ARR from 15.3 to 0.6 (ng/dL)/(mU/L) as shown in FIG. 2A and Table 4. Dexfadrostat phosphate treatment decreased mean 24-h aSBP by 10.7 mmHg from baseline to day 56 as shown in FIG. 2B and Table 5 (p<0.0001 for both median plasma ARR reduction and aSBP reduction; all dose groups combined).

TABLE 4
Change in Mean ARR (ng/dL)/(mU/L) From Baseline to Day 56

| All Doses | n | Mean | SD | Q1 | Median | Q3 | Min | Max |
|---|---|---|---|---|---|---|---|---|
| Day 1 | 35 | 17.58 | 15.19 | 5.32 | 15.27 | 25.89 | 0.3 | 68.6 |
| Day 56 | 35 | 2.18 | 3.29 | 0.26 | 0.63 | 3.03 | 0.1 | 13.4 |
| Change | 35 | −15.40 | 13.93 | 13.93 | −11.76 | −4.90 | −63.10 | −0.10 |

TABLE 5
Change in Mean aSBP (mmHg) From Baseline to Day 56

| All Doses | n | Mean | SD | Q1 | Median | Q3 | Min | Max |
|---|---|---|---|---|---|---|---|---|
| Day 1 | 35 | 142.62 | 14.177 | 134.77 | 143.4 | 149.95 | 115.8 | 177.67 |
| Day 56 | 35 | 131.87 | 13.387 | 124.03 | 130.41 | 137.61 | 96.99 | 164.65 |
| Change | 35 | −10.74 | 8.90 | −16.90 | −12.23 | −6.72 | −31.38 | 14.76 |

### Safety

During the 70-day study period, TEAEs were reported in 16 patients. Seven patients had a TEAE that was considered related to or possibly related to dexfadrostat phosphate, and five had events that were related to or possibly related to anti-hypertensive treatments received during the study (Table 6). No serious TEAEs, fatal TEAEs or AEs leading to treatment discontinuation were reported. Adverse events of special interest (AESI) were hyperkalemia, headache, dizziness, and orthostatic hypotension. AESIs were reported in four patients; all were headaches and two were considered to be treatment-related or possibly treatment-related.

TABLE 6
Treatment Emergent/Related Adverse Events

| Event | N = 35 (%) | |
|---|---|---|
| Any TEAE | 16 | (45.7) |
| Serious TEAE | 0 | (0) |
| TRAE | 7 | (20.0) |

| Breakdown of TRAE and possibly TRAE | Number of events |
|---|---|
| Vertigo | 1 |
| Gastritis | 1 |
| Nausea | 1 |
| Headache | 2 |
| Amenorrhea | 1 |
| Breast tenderness | 1 |
| Polymenorrhea | 1 |
| Cough | 1 |

### Example 2

### A Double-Blind, Randomized, Phase 2 Trial in Patients with Primary Aldosteronism to Investigate the Efficacy of Dexfadrostat Phosphate to Correct Biochemical (High Plasma ARR) Disease Parameters Caused by Different Pathologies

Upon investigator discretion, the diagnosis of PA was extended according to Endocrine Society Clinical Practice Guidelines by subjecting patients to a CT scan to image the presence of any adrenal adenoma or nodules and subsequently to adrenal vein sampling (AVS) to lateralize an aldosterone-producing adrenal gland. Per guideline recommendation, unilateral adenoma or nodules are generally surgically removed by adrenalectomy because this pathology is considered more severe and medically difficult to manage.

A per-patient analysis of study results on biochemical correction of high plasma ARR levels investigated whether patients with defined pathologies such a unilateral adenoma or nodules respond differently to dexfadrostat phosphate than undefined underlying pathologies such as bilateral nodules or hyperplasia or even normal appearing glands (see Table 7).

TABLE 7
Change in ARR ((ng/dL)/(mU/L)) from Baseline to Day 56 by
Dose of Dexfadrostat Phosphate and by PA Causing Pathology

| | PA subtyping | | | | ARR (ng*L/dL*mU) | | | | |
|---|---|---|---|---|---|---|---|---|---|
| Patient | CT Scan | CT Result | AVS | AVS Result | Day 1 | Day 14 | Day 28 | Day 42 | Day 56 |
| 4 mg ARR | | | | | | | | | |
| 1 | Y | Nodule R | N | n/a | 18.0 | 0.7 | 3.1 | 3.8 | 3.7 |
| 5 | Y | normal | N | n/a | 24.8 | 0.4 | 1.1 | 1.7 | 0.9 |
| 8 | N | n/a | N | n/a | 68.6 | 7.3 | 6.6 | 6.3 | 5.5 |
| 14 | Y | normal | N | n/a | 50.5 | 8.6 | 4.0 | 8.5 | 4.1 |
| 18 | N | n/a | N | n/a | 25.9 | 7.3 | 1.2 | 0.5 | 0.4 |
| 21 | Y | Adenoma L | N | n/a | 4.5 | 0.4 | 1.5 | 0.7 | 1.4 |
| 22 | Y | Adenoma R/HP L | N | n/a | 28.9 | 12.9 | 1.9 | 2.2 | 13.2 |
| 25 | Y | normal | N | n/a | 2.4 | 1.1 | 0.5 | 0.3 | 0.6 |
| 28 | Y | normal | N | n/a | 6.8 | 0.6 | 1.6 | 2.0 | 1.2 |
| 30 | Y | Adenoma L | N | n/a | 15.3 | 6.5 | 4.2 | 2.9 | 6.0 |
| 8 mg ARR | | | | | | | | | |
| 2 | Y | normal | Y | unilateral R | 11.3 | 3.4 | 2.0 | 1.1 | 0.6 |
| 6 | Y | Nodule L | N | n/a | 29.6 | 0.5 | 1.3 | 1.9 | 0.9 |
| 7 | N | n/a | N | n/a | 35.6 | 1.3 | 6.1 | 2.5 | 5.2 |
| 11 | N | n/a | N | n/a | 3.3 | 0.3 | 0.2 | 0.1 | 0.1 |
| 12 | N | n/a | N | n/a | 7.0 | 0.2 | 0.4 | 0.4 | 0.2 |
| 16 | Y | Hyperplasia L | N | n/a | 21.1 | 2.6 | 0.8 | 1.2 | 2.9 |
| 20 | N | n/a | N | n/a | 7.3 | 0.1 | 0.4 | 0.2 | 0.2 |
| 24 | Y | Abnormal L | Y | unilateral L | 5.6 | 0.4 | 3.2 | 0.8 | 0.6 |
| 27 | Y | Hyperplasia L | Y | unilateral R | 0.3 | 0.2 | 0.2 | 0.2 | 0.2 |
| 29 | N | n/a | N | n/a | 2.6 | 0.1 | 0.3 | 0.2 | 0.3 |
| 33 | Y | normal | N | ADX 2020-1 | 13.9 | 0.4 | 0.3 | 0.1 | 0.2 |
| 35 | Y | Adenoma L | Y | unilateral L | 1.4 | 0.1 | 0.2 | 0.1 | 0.3 |
| 12 mg ARR | | | | | | | | | |
| 3 | Y | Nodule R | Y | ? | 34.4 | 10.3 | 6.4 | 4.8 | 5.2 |
| 4 | Y | Nodule L | Y | Adenoma L | 17.0 | 4.6 | 3.4 | 1.9 | 3.0 |
| 9 | Y | Nodule L | Y | Adenoma L | 21.5 | 0.3 | 0.1 | 0.6 | 0.7 |
| 10 | Y | Nodule L | Y | Bilateral | 3.2 | 0.2 | 0.2 | 0.3 | 0.1 |
| 13 | Y | Nodule L | N | n/a | 10.1 | 0.3 | 0.5 | 0.7 | 0.5 |
| 15 | Y | Nodule L/HP R | Y | unilateral L | 17.8 | 2.7 | 1.1 | 0.9 | 0.4 |
| 17 | N | n/a | N | n/a | 34.9 | 4.2 | 4.0 | 3.0 | 1.1 |
| 19 | Y | Adenoma R | N | n/a | 25.2 | 2.4 | 4.4 | 1.0 | 13.4 |
| 23 | N | n/a | N | n/a | 19.5 | 1.0 | 18.7 | 19.9 | 2.1 |
| 26 | Y | normal | N | n/a | 3.3 | 0.1 | 0.1 | 0.1 | 0.2 |
| 31 | Y | normal | Y | failure | 29.5 | 0.2 | 0.3 | 0.1 | 0.3 |
| 32 | Y | normal | N | n/a | 5.3 | 0.2 | 0.1 | 0.3 | 0.4 |
| 34 | Y | Adenoma R | Y | unilateral R | 9.0 | 0.3 | 0.6 | 0.5 | 0.6 |

Surprisingly, patients with unilateral disease such as patients 1, 21, 22, 30 treated with 4 mg dexfadrostat phosphate; and patients 6, 16, 24, 27, 35 treated with 8 mg dexfadrostat phosphate and patients 3, 4, 9, 13, 15, 19 and 34 treated with 12 mg dexfadrostat phosphate demonstrated a similar response compared to undefined or bilateral disease in the respective dose groups. A representation of the combined doses demonstrates that dexfadrostat phosphate normalized the ARR in patients with unilateral disease (FIG. 3A) as well as patients with bilateral or undefined disease (FIG. 3B).

### Example 3

### A Double-Blind, Randomized, Phase 2 Trial in Patients with Primary Aldosteronism to Investigate the Dose-Dependent Efficacy of Dexfadrostat Phosphate to Correct Biochemical (High Plasma ARR) and Clinical (High Blood Pressure) Disease Parameters

Secondary endpoints of the study assessed whether dexfadrostat phosphate showed dose-dependent efficacy in correcting the biochemical (ARR) and clinical (blood pressure) parameters of primary aldosteronism. The daily doses of 4 mg, 8 mg and 12 mg dexfadrostat phosphate were derived from the preceding clinical phase 1 study (PCT/EP2019/061283; see WO 2019/211394). Analysis of the individual dose arms revealed that all three doses mediated significant reductions (p<0.0001) in ARR from baseline to day 56 (see Table 8 and FIG. 4A, 4B, 4C). Surprisingly, the 4 mg dose was revealed to be only slightly less efficacious than the 8 mg dose. In contrast, the 12 mg dose was not more potent than the 8 mg dose indicating that the maximal drug efficacy was reached with the 8 mg dose.

TABLE 8
Change in ARR ((ng/dL)/(mU/L)) from Baseline
to Day 56 by Dose of Dexfadrostat Phosphate

| | n | mean | SD | Q1 | median | Q3 | Min | Max |
|---|---|---|---|---|---|---|---|---|
| 4 mg ARR | | | | | | | | |
| Day 1 | 10 | 24.56 | 21.011 | 6.84 | 21.37 | 28.88 | 2.4 | 68.6 |
| Day 56 | 10 | 3.70 | 3.94 | 0.92 | 2.52 | 5.51 | 0.4 | 13.2 |
| Change | 10 | −20.87 | 19.942 | −25.48 | −14.98 | −5.67 | −63.1 | −1.8 |
| 8 mg ARR | | | | | | | | |
| Day 1 | 12 | 11.58 | 11.501 | 2.92 | 7.18 | 17.51 | 0.3 | 35.6 |
| Day 70 | 12 | 4.19 | 4.358 | 1.27 | 2.46 | 6.31 | 0.5 | 14.0 |
| Change | 12 | −10.61 | 10.322 | −15.97 | −6.99 | −2.74 | −30.5 | −0.1 |
| 12 mg ARR | | | | | | | | |
| Day 1 | 13 | 17.75 | 11.164 | 8.97 | 17.8 | 25.16 | 3.2 | 34.9 |
| Day 70 | 13 | 9.3 | 10.482 | 1.8 | 13.72 | 13.72 | 0.6 | 32.1 |
| Change | 13 | −15.61 | 10.3 | −20.8 | −13.97 | −8.37 | −33.9 | −3.1 |

In each dose arm, treatment with dexfadrostat phosphate and the suppression of the ARR resulted in a significant reduction (p<0.001) in 24-hour aSBP (see Table 9 and FIG. 4D, 4E, 4F). Surprisingly, the 4 mg dose was shown to be only slightly less efficacious than the 8 mg dose. In contrast, the 12 mg dose was not more potent than the 8 mg dose indicating that the maximal drug efficacy was reached with the 8 mg dose.

TABLE 9
Change in Mean 24-h aSBP (mmHg) from Baseline
to Day 56 by Dose of Dexfadrostat Phosphate

| | n | mean | SD | Q1 | median | Q3 | Min | Max |
|---|---|---|---|---|---|---|---|---|
| 4 mg aSBP | | | | | | | | |
| Day 1 | 10 | 136.84 | 15.92 | 123.85 | 138.38 | 144.41 | 115.8 | 168.7 |
| Day 56 | 10 | 128.74 | 13.903 | 124.71 | 129.43 | 137.61 | 97 | 150 |
| Change | 10 | −8.10 | 9.51 | −18.35 | −8.17 | −3.98 | −19.70 | 11.40 |
| 8 mg aSBP | | | | | | | | |
| Day 1 | 12 | 144.88 | 13.946 | 137.47 | 144.76 | 154.83 | 120.9 | 167 |
| Day 56 | 12 | 130.93 | 11.692 | 121.41 | 129.43 | 138.5 | 118 | 150.5 |
| Change | 12 | −13.95 | 8.14 | −18.95 | −13.33 | −7.47 | −31.40 | −2.80 |
| 12 mg aSBP | | | | | | | | |
| Day 1 | 13 | 144.96 | 12.735 | 135.28 | 146.87 | 149.95 | 128 | 177.7 |
| Day 56 | 13 | 135.15 | 14.725 | 126.71 | 130.82 | 136.6 | 115.5 | 164.6 |
| Change | 13 | −9.81 | 8.89 | −13.82 | −12.43 | −8.66 | −19.10 | 14.80 |

### Example 4

### A Double-Blind, Randomized, Phase 2 Trial in Patients with Primary Aldosteronism to Investigate the Dose-Dependent Efficacy of Dexfadrostat Phosphate to Correct a Biochemical (High Urinary Aldosterone Content) Disease Parameter

An exploratory endpoint of the study assessed whether dexfadrostat phosphate demonstrated dose-dependent efficacy in correcting the high aldosterone content in urine, a disease parameter of primary aldosteronism but also other cardiovascular diseases. The daily doses of 4 mg, 8 mg and 12 mg dexfadrostat phosphate were derived from the preceding clinical phase 1 study (PCT/EP2019/061283; see WO 2019/211394). Analysis of the individual dose arms revealed that all three doses mediated significant reductions (p<0.0001) in tetrahydroaldosterone (THA) content, the main metabolite of aldosterone in urine in 24-hour urine samples collected at the end of active treatment compared to baseline samples (see Table 10 and FIG. 4G, 4H, 4I). Surprisingly, the 4 mg dose was revealed to be only slightly less efficacious than the 8 mg dose. In contrast, the 12 mg dose was not more potent than the 8 mg dose indicating that the maximal drug efficacy was reached with the 8 mg dose.

TABLE 10
Change in urine tetrahydroaldosterone (THA) content (24 μg/24-
hour) from Baseline to Day 56 by Dose of Dexfadrostat Phosphate

| | n | mean | SD | Q1 | median | Q3 | Min | Max |
|---|---|---|---|---|---|---|---|---|
| 4 mg uTHA | | | | | | | | |
| Day 1 | 10 | 78.90 | 41.81 | 54.00 | 80.00 | 102.00 | 11.00 | 157.00 |
| Day 56 | 10 | 12.00 | 6.70 | 5.00 | 11.50 | 17.00 | 5.00 | 23.00 |
| Change | 10 | −66.90 | 40.56 | −86.00 | −61.00 | −45.00 | −152.00 | −6.00 |
| 8 mg uTHA | | | | | | | | |
| Day 1 | 12 | 71.67 | 56.81 | 41.00 | 59.50 | 76.00 | 15.00 | 226.00 |
| Day 56 | 12 | 8.67 | 10.12 | 2.00 | 5.00 | 10.00 | 2.00 | 36.00 |
| Change | 12 | −63.00 | 56.57 | −74.00 | −50.00 | −28.50 | −224.00 | −12.00 |
| 12 mg uTHA | | | | | | | | |
| Day 1 | 13 | 67.38 | 28.10 | 56.00 | 69.00 | 76.00 | 11.00 | 108.00 |
| Day 56 | 13 | 9.08 | 11.27 | 3.00 | 5.00 | 9.00 | 2.00 | 44.00 |
| Change | 13 | −58.31 | 29.25 | −71.00 | −66.00 | −41.00 | −103.00 | −8.00 |

### Example 5

### A Double-Blind, Randomized, Phase 2 Trial in Patients with Primary Aldosteronism to Investigate the Time- and Withdrawal-Dependent Efficacy of Dexfadrostat Phosphate to Correct a Biochemical (Plasma ARR and Potassium) and Clinical (Blood Pressure) Disease Parameter

Secondary endpoints of the study assessed whether dexfadrostat phosphate showed dose and time-dependent efficacy in correcting the biochemical (plasma ARR and potassium) and clinical (blood pressure) parameters of primary aldosteronism. The daily doses of 4 mg, 8 mg and 12 mg dexfadrostat phosphate were derived from the preceding clinical phase 1 study (PCT/EP2019/061283; see WO 2019/211394). Analysis of the individual dose arms revealed that all three doses mediated rapid and significant reductions (p<0.0001) in ARR from baseline to day 14, day 28, day 42 and day 56 clinic visits (see Table 11 and FIG. 5A, 5D). The ARR suppression remained sustained over the entire active treatment period. Upon drug withdrawal, ARR values returned towards baseline values. Surprisingly, the 4 mg dose was revealed to be only slightly less efficacious than the 8 mg dose. In contrast, the 12 mg dose was not more potent than the 8 mg dose indicating that the maximal drug efficacy was reached with the 8 mg dose.

TABLE 11
Change in ARR ((ng/dL)/(mU/L)) from Baseline throughout Day 56 and
after Withdrawal at Day 70 by Dose of Dexfadrostat Phosphate

| | n | mean | SD | Q1 | median | Q3 | Min | Max |
|---|---|---|---|---|---|---|---|---|
| 4 mg | | | | | | | | |
| Day 1 | 10 | 24.56 | 21.011 | 6.84 | 21.37 | 28.88 | 2.4 | 68.6 |
| Day 14 | 10 | 4.57 | 4.475 | 0.62 | 3.76 | 7.33 | 0.4 | 12.9 |
| Day 28 | 10 | 2.57 | 1.871 | 1.23 | 1.76 | 4.04 | 0.5 | 6.6 |
| Day 42 | 10 | 2.87 | 2.676 | 0.73 | 2.07 | 3.75 | 0.3 | 8.5 |
| Day 56 | 10 | 3.70 | 3.94 | 0.92 | 2.52 | 5.51 | 0.4 | 13.2 |
| Day 70 | 10 | 12.34 | 8.738 | 2.98 | 11.47 | 20.97 | 1.5 | 25.7 |
| 8 mg | | | | | | | | |
| Day 1 | 12 | 11.58 | 11.501 | 2.92 | 7.18 | 17.51 | 0.3 | 35.6 |
| Day 14 | 12 | 0.8 | 1.085 | 0.18 | 0.31 | 0.9 | 0.1 | 3.4 |
| Day 28 | 12 | 1.26 | 1.774 | 0.23 | 0.4 | 1.65 | 0.2 | 6.1 |
| Day 42 | 12 | 0.74 | 0.801 | 0.16 | 0.29 | 1.16 | 0.1 | 2.5 |
| Day 56 | 12 | 0.96 | 1.523 | 0.22 | 0.27 | 0.77 | 0.1 | 5.2 |
| Day 70 | 12 | 4.19 | 4.358 | 1.27 | 2.46 | 6.31 | 0.5 | 14.0 |

In each dose arm, the suppression of the ARR resulted in a rapid and significant (p<0.001) increase in plasma potassium concentrations (see Table 12 and FIG. 5C, 5F). The potassium increase remained sustained over the entire active treatment period. Upon drug withdrawal, potassium values returned towards baseline levels. Surprisingly, the 4 mg dose was as efficacious as the 8 mg dose. The 12 mg dose provided no additional efficacy.

TABLE 12
Change in plasma potassium (nmol/L) from Baseline
throughout Day 56 and after Withdrawal at Day
70 by Dose of Dexfadrostat Phosphate

| | n | mean | SD | Q1 | median | Q3 | Min | Max |
|---|---|---|---|---|---|---|---|---|
| 4 mg | | | | | | | | |
| Day 1 | 10 | 3.36 | 0.448 | 3.20 | 3.35 | 3.60 | 2.40 | 4.00 |
| Day 14 | 10 | 4.27 | 0.422 | 3.90 | 4.35 | 4.50 | 3.50 | 5.00 |
| Day 28 | 10 | 3.85 | 0.288 | 3.60 | 3.85 | 4.10 | 3.40 | 4.30 |
| Day 42 | 10 | 4.07 | 0.430 | 4.00 | 4.15 | 4.30 | 3.20 | 4.80 |
| Day 56 | 10 | 4.10 | 0.330 | 3.90 | 4.20 | 4.30 | 3.50 | 4.60 |
| Day 70 | 10 | 3.58 | 0.516 | 3.40 | 3.60 | 4.00 | 2.40 | 4.10 |
| 8 mg | | | | | | | | |
| Day 1 | 11 | 3.55 | 0.311 | 3.40 | 3.50 | 3.80 | 3.00 | 4.10 |
| Day 14 | 11 | 4.32 | 0.264 | 4.10 | 4.30 | 4.60 | 3.90 | 4.70 |
| Day 28 | 11 | 4.04 | 0.359 | 3.70 | 4.00 | 4.30 | 3.60 | 4.70 |
| Day 42 | 11 | 3.99 | 0.234 | 3.90 | 3.90 | 4.20 | 3.70 | 4.40 |
| Day 56 | 11 | 4.10 | 0.210 | 3.90 | 4.10 | 4.20 | 3.80 | 4.50 |
| Day 70 | 11 | 3.76 | 0.264 | 3.50 | 3.80 | 4.00 | 3.40 | 4.10 |

In each dose arm, the suppression of the ARR resulted also in a rapid and significant (p≤0.001) decrease in office blood pressure (see Table 13 and FIG. 5B, 5E). The blood pressure decrease increase remained sustained over the entire active treatment period. Upon drug withdrawal, blood pressure values returned towards baseline levels. Surprisingly, the 4 mg dose was almost as efficacious as the 8 mg dose. The 12 mg dose provided no additional clinical benefits.

TABLE 13
Change in office blood pressure (mmHg) from Baseline throughout Day
56 and after Withdrawal at Day 70 by Dose of Dexfadrostat Phosphate

| | n | mean | SD | Q1 | median | Q3 | Min | Max |
|---|---|---|---|---|---|---|---|---|
| 4 mg | | | | | | | | |
| Day 1 | 10 | 146.95 | 14.29 | 139.50 | 144.00 | 154.50 | 127.50 | 179.50 |
| Day 14 | 10 | 134.55 | 19.30 | 125.50 | 136.25 | 147.50 | 101.50 | 164.00 |
| Day 28 | 10 | 133.70 | 19.03 | 118.00 | 130.50 | 142.00 | 110.00 | 164.00 |
| Day 42 | 10 | 134.90 | 17.69 | 123.50 | 132.75 | 147.50 | 105.00 | 160.00 |
| Day 56 | 10 | 133.00 | 12.90 | 123.50 | 133.75 | 146.50 | 113.00 | 148.50 |
| Day 70 | 10 | 142.20 | 11.00 | 131.50 | 145.25 | 151.00 | 125.00 | 156.00 |
| 8 mg | | | | | | | | |
| Day 1 | 12 | 148.00 | 6.25 | 143.50 | 147.00 | 153.50 | 139.00 | 158.00 |
| Day 14 | 12 | 137.88 | 12.91 | 132.50 | 137.50 | 142.00 | 116.00 | 167.00 |
| Day 28 | 12 | 135.08 | 9.75 | 130.75 | 133.25 | 143.25 | 114.00 | 149.50 |
| Day 42 | 12 | 137.83 | 9.84 | 130.25 | 134.50 | 145.50 | 126.00 | 156.50 |
| Day 56 | 12 | 134.42 | 8.08 | 128.75 | 135.75 | 140.00 | 122.00 | 149.00 |
| Day 70 | 12 | 142.46 | 12.52 | 135.00 | 139.75 | 152.75 | 122.00 | 163.00 |

### Example 6

### A Double-Blind. Randomized. Phase 2 Trial in Patients with Primary Aldosteronism to Investigate Blood Potassium Levels. Blood Aldosterone Levels and 24-Hour Urine Aldosterone Content

TABLE 14
Change in Blood Potassium Concentration in Patient
Segments Defined by Initial Blood Potassium Levels
Plasma Potassium Concentration at
Baseline (Day 1): <3.6 mmol/L

| | Treatment | | | | | |
|---|---|---|---|---|---|---|
| | 4 mg QD | | | | | Placebo |
| Visits | Day 1 | Day 14 | Day 28 | Day 42 | Day 56 | Day 70 |
| Mean | 3.1 | 4.1 | 3.8 | 3.9 | 4.0 | 3.3 |
| SD | 0.4 | 0.4 | 0.3 | 0.4 | 0.3 | 0.5 |
| | Treatment | | | | | |
| | 8 mg QD | | | | | Placebo |
| Visits | Day 1 | Day 14 | Day 28 | Day 42 | Day 56 | Day 70 |
| Mean | 3.3 | 4.2 | 3.9 | 4.0 | 4.0 | 3.7 |
| SD | 0.2 | 0.5 | 0.3 | 0.3 | 0.2 | 0.2 |
| | Treatment | | | | | |
| | 12 mg QD | | | | | Placebo |
| Visits | Day 1 | Day 14 | Day 28 | Day 42 | Day 56 | Day 70 |
| Mean | 3.3 | 3.9 | 4.0 | 4.1 | 3.8 | 3.7 |
| SD | 0.2 | 0.1 | 0.3 | 0.3 | 0.3 | 0.4 |

Plasma Potassium Concentration at
Baseline (Day 1): ≥3.6 mmol/L

| | Treatment | | | | | |
|---|---|---|---|---|---|---|
| | 4 mg QD | | | | | Placebo |
| Visits | Day 1 | Day 14 | Day 28 | Day 42 | Day 56 | Day 70 |
| Mean | 3.8 | 4.6 | 4.0 | 4.3 | 4.3 | 4.0 |
| SD | 0.2 | 0.3 | 0.2 | 0.4 | 0.2 | 0.2 |
| | Treatment | | | | | |
| | 8 mg QD | | | | | Placebo |
| Visits | Day 1 | Day 14 | Day 28 | Day 42 | Day 56 | Day 70 |
| Mean | 3.8 | 4.3 | 4.1 | 3.9 | 4.1 | 3.9 |
| SD | 0.2 | 0.3 | 0.4 | 0.2 | 0.3 | 0.3 |
| | Treatment | | | | | |
| | 12 mg QD | | | | | Placebo |
| Visits | Day 1 | Day 14 | Day 28 | Day 42 | Day 56 | Day 70 |
| Mean | 3.9 | 4.0 | 4.1 | 4.0 | 3.9 | 3.7 |
| SD | 0.3 | 0.5 | 0.3 | 0.4 | 0.6 | 0.6 |

Plasma Potassium Concentration at
Baseline (Day 1): ≤3.0 mmol/L

| | Treatment | | | | | |
|---|---|---|---|---|---|---|
| | all doses QD | | | | | Placebo |
| Visits | Day 1 | Day 14 | Day 28 | Day 42 | Day 56 | Day 70 |
| Mean | 2.9 | 3.9 | 3.9 | 4.0 | 3.9 | 3.5 |
| SD | 0.3 | 0.3 | 0.3 | 0.5 | 0.4 | 0.7 |

Plasma Potassium Concentration Changes
in Presence of Potassium Supplements

| | Treatment | | | | | |
|---|---|---|---|---|---|---|
| | all doses QD | | | | | Placebo |
| Visits | Day 1 | Day 14 | Day 28 | Day 42 | Day 56 | Day 70 |
| Mean | 3.3 | 4.1 | 4.0 | 4.1 | 4.1 | 3.8 |
| SD | 0.4 | 0.4 | 0.3 | 0.3 | 0.2 | 0.2 |

Plasma Potassium Concentration at Baseline
(Day 1) ≤3.0 mmol/L Despite Potassium Supplements

| | Treatment | | | | | |
|---|---|---|---|---|---|---|
| | all doses QD | | | | | Placebo |
| Visits | Day 1 | Day 14 | Day 28 | Day 42 | Day 56 | Day 70 |
| Mean | 3.2 | 4.0 | 4.1 | 4.2 | 4.0 | 3.8 |
| SD | 0.4 | 0.3 | 0.3 | 0.3 | 0.2 | 0.3 |

TABLE 15
Change in Plasma Aldosterone Concentration in Patient
Segments Defined by Initial Plasma Aldosterone Levels
Plasma Aldosterone Concentration
Range at Baseline (Day 1): 400-600 pmol/L

| | Treatment | | | | | |
|---|---|---|---|---|---|---|
| | 4 mg QD | | | | | Placebo |
| Visits | Day 1 | Day 14 | Day 28 | Day 42 | Day 56 | Day 70 |
| Mean | 433 | 155 | 100 | 117 | 141 | 425 |
| SD | 61 | 39 | 15 | 45 | 34 | 138 |
| | Treatment | | | | | |
| | 8 mg QD | | | | | Placebo |
| Visits | Day 1 | Day 14 | Day 28 | Day 42 | Day 56 | Day 70 |
| Mean | 404 | 70 | 70 | 47 | 58 | 300 |
| SD | 175 | 30 | 41 | 23 | 25 | 213 |
| | Treatment | | | | | |
| | 12 mg QD | | | | | Placebo |
| Visits | Day 1 | Day 14 | Day 28 | Day 42 | Day 56 | Day 70 |
| Mean | 431 | 63 | 132 | 142 | 61 | 297 |
| SD | 83 | 37 | 19 | 206 | 28 | 212 |

Plasma Aldosterone Concentration Range
at Baseline (Day 1): 600-1000 pmol/L

| | Treatment | | | | | |
|---|---|---|---|---|---|---|
| | 4 mg QD | | | | | Placebo |
| Visits | Day 1 | Day 14 | Day 28 | Day 42 | Day 56 | Day 70 |
| Mean | 728 | 186 | 248 | 114 | 250 | 325 |
| SD | 62 | 165 | 308 | 62 | 147 | 116 |
| | Treatment | | | | | |
| | 8 mg QD | | | | | Placebo |
| Visits | Day 1 | Day 14 | Day 28 | Day 42 | Day 56 | Day 70 |
| Mean | 805 | 83 | 120 | 123 | 131 | 423 |
| SD | 118 | 26 | 32 | 51 | 130 | 286 |
| | Treatment | | | | | |
| | 12 mg QD | | | | | Placebo |
| Visits | Day 1 | Day 14 | Day 28 | Day 42 | Day 56 | Day 70 |
| Mean | 912 | 123 | 104 | 85 | 82 | 466 |
| SD | 69 | 114 | 62 | 44 | 50 | 318 |

Plasma Aldosterone Concentration Range
at Baseline (Day 1): 1000-2500 pmol/L

| | Treatment | | | | | |
|---|---|---|---|---|---|---|
| | 4 mg QD | | | | | Placebo |
| Visits | Day 1 | Day 14 | Day 28 | Day 42 | Day 56 | Day 70 |
| Mean | 2005 | 816 | 531 | 629 | 589 | 1393 |
| SD | 474 | 295 | 400 | 480 | 413 | 658 |
| | Treatment | | | | | |
| | 8 mg QD | | | | | Placebo |
| Visits | Day 1 | Day 14 | Day 28 | Day 42 | Day 56 | Day 70 |
| Mean | 1080 | 27 | 57 | 27 | 27 | 395 |
| | Treatment | | | | | |
| | 12 mg QD | | | | | Placebo |
| Visits | Day 1 | Day 14 | Day 28 | Day 42 | Day 56 | Day 70 |
| Mean | 1410 | 111 | 74 | 71 | 349 | 1264 |
| SD | 341 | 75 | 48 | 39 | 448 | 950 |

TABLE 16
Change in Urine Aldosterone Concentration in Patient
Segments Defined by Initial Urine Aldosterone Levels
Urine Aldosterone Content Range at Baseline (Day 1):
10-50 μg/24-hour

Treatment
4 mg QD
Visits

| | Day 1 | Day 56 |
|---|---|---|
| Mean | 26 | 6 |
| SD | 21 | 1 |

Treatment
8 mg QD
Visits

| | Day 1 | Day 56 |
|---|---|---|
| Mean | 33 | 8 |
| SD | 15 | 7 |

Treatment
12 mg QD
Visits

| | Day 1 | Day 56 |
|---|---|---|
| Mean | 29 | 3 |
| SD | 17 | 0 |

Urine Aldosterone Content Range at Baseline (Day 1):
50-100 μg/24-hour

Treatment
4 mg QD
Visits

| | Day 1 | Day 56 |
|---|---|---|
| Mean | 74 | 15 |
| SD | 21 | 5 |

Treatment
8 mg QD
Visits

| | Day 1 | Day 56 |
|---|---|---|
| Mean | 69 | 5 |
| SD | 9 | 4 |

Treatment
12 mg QD
Visits

| | Day 1 | Day 56 |
|---|---|---|
| Mean | 68 | 12 |
| SD | 8 | 15 |

Urine Aldosterone Content Range at Baseline (Day 1):
100-250 μg/24-hour

Treatment
4 mg QD
Visits

| | Day 1 | Day 56 |
|---|---|---|
| Mean | 123 | 13 |
| SD | 30 | 8 |

Treatment
8 mg QD
Visits

| | Day 1 | Day 56 |
|---|---|---|
| Mean | 69 | 5 |
| SD | 9 | 4 |

Treatment
12 mg QD
Visits

| | Day 1 | Day 56 |
|---|---|---|
| Mean | 68 | 12 |
| SD | 8 | 15 |

1. A composition for use in the treatment of a disease or disorder, wherein said composition is administered once daily to a subject, preferably a human, in need thereof wherein said composition comprises a compound which is (R)-(+)-5-(p-cyanophenyl)-5,6,7,8-tetrahydroimidazo[1,5-a]pyridine or a pharmaceutically acceptable salt thereof, wherein said compound has an enantiomeric excess (ee) of the (R) form higher than or equal to about 99%.

2. The composition for use of claim 1, wherein said composition is administered once daily to a subject in need thereof for at least eight weeks.

3. The composition for use of any of claim 1 or 2, wherein said disease or disorder is a chronic disease or disorder, preferably a chronic cardiovascular disease or disorder.

4. The composition for use of any of the preceding claims, wherein said composition is administered once daily to said subject at a dosage from about 1 mg to about 12 mg of said compound.

5. The composition for use of any of the preceding claims, wherein said compound is administered at a dosage of about 4 mg.

6. The composition for use of any of any of claims 1-4, wherein said compound is administered at a dosage of about 8 mg.

7. The composition for use of any of the preceding claims, wherein said compound is (R)-(+)-5-(p-cyanophenyl)-5,6,7,8-tetrahydroimidazolium[1,5-a]pyridine dihydrogen phosphate, preferably wherein said (R)-(+)-5-(p-cyanophenyl)-5,6,7,8-tetrahydroimidazolium[1,5-a]pyridine dihydrogen phosphate has an enantiomeric excess (ee) of the (R) form higher than or equal to about 99.5%, more preferably higher than or equal to about 99.9%.

8. The composition for use of any of the preceding claims, wherein said disease or disorder is selected from the group consisting of primary aldosteronism, secondary aldosteronism, heart failure, chronic renal failure, hypertension, restenosis, obesity, nephropathy, post-myocardial infarction, renal fibrosis, coronary heart disease, sodium retention, water retention, hypokalemia, hypomagnesaemia, hypertension, left ventricular hypertrophy, cardiac fibrosis, cardiovascular damage, suppression of plasma renin, kidney fibrosis, arrhythmias, nephropathy, edema, hypokalemia-caused muscle weakness, cardiac fibrillation and weakened cardiac muscle contraction;
preferably wherein said disease or disorder is selected from the group consisting of primary aldosteronism, secondary aldosteronism, heart failure, chronic renal failure, hypertension, restenosis, obesity, nephropathy, post-myocardial infarction, renal fibrosis, coronary heart disease, sodium retention, water retention, hypokalemia, hypomagnesaemia, hypertension, left ventricular hypertrophy, cardiac fibrosis, cardiovascular damage, suppression of plasma renin, kidney fibrosis, arrhythmias, nephropathy, and edema.

9. The composition for use of any of the preceding claims, said disease or disorder is primary aldosteronism.

10. The composition or use of any of the preceding claims, wherein said disease or disorder is bilateral primary aldosteronism.

11. The composition of any of claims 1-9, wherein said disease or disorder is unilateral primary aldosteronism, preferably wherein said unilateral primary aldosteronism is caused by unilateral adenoma, unilateral nodules, or unilateral hyperplasia.

12. The composition for use of any of claims 1-8, wherein said disease or disorder is essential hypertension and/or resistant hypertension, preferably wherein said disease or disorder is essential hypertension and/or resistant hypertension with accompanying hypokalemia.

13. The composition for use of any of claims 1-8, wherein said disease or disorder is hypokalemia.

14. The composition for use of any of the above-claims, wherein said once daily dose does not require dose adjustment.

15. The composition for use of any of the above-claims, wherein said subject exhibits a clinical response within about 14 days of treatment, preferably wherein said clinical response is selected from reduction in plasma aldosterone levels, normalization of plasma aldosterone levels, reduction of blood pressure, normalization of blood pressure, reduction of hypokalemia, normalization of potassium concentration, and reduction of urinary THA.
